(19) United States
(12) Patent Application Publication
(10) Pub. No.: US 2019/0348158 A1
(43) Pub. Date: Nov. 14, 2019

(54) SYSTEMS AND METHODS FOR MANAGING DATA PRIVACY

(71) Applicant: Michigan Health Information Network Shared Services, East Lansing, MI (US)

(72) Inventors: Jeff Livesay, Pinckney, MI (US); Tim Pletcher, Mount Pleasant, MI (US)

(21) Appl. No.: 15/977,690

(22) Filed: May 11, 2018

(51) Int. Cl. G16H 10/60; G06F 21/62; G16H 15/00

(52) U.S. Cl. CPC G16H 10/60; G16H 15/00; G06F 21/6245

(57) ABSTRACT

This disclosure provides systems and methods for managing data privacy. Consent information can be received including a first patient identifier, a plurality of health organization identifiers, and an indication that a patient corresponding to the first patient identifier consents to sharing of health information by a plurality of health organizations corresponding to the plurality of health organization identifiers. A consent record can be stored in a database based on the consent information. A query can be received from a data sharing organization including a second patient identifier. It can be determined that data sharing organization has permission to share health information related to the patient, based on the query and the consent record. A response to the query can be generated based on the determination that the data sharing organization has permission to share health information related to the patient. The response can be transmitted to the data sharing organization.

### BACKGROUND

A large volume of medical information can be stored electronically for individuals or populations. For example, information relating to patients' diagnosed conditions, prescribed medications, health outcomes, medical procedures undergone, and other information can be recorded across a wide variety of computing devices, such as servers maintained by different health organizations. It may be beneficial for various health organizations to share certain health data relating to a patient in order to provide medical personnel with more complete health information for the patient. However, health organizations must be careful to protect health information and other sensitive information in order to maintain patient privacy and to comply with both federal and state regulations. As a result, requesting and obtaining medical information electronically across a distributed healthcare system can be challenging.

### SUMMARY

One aspect of this disclosure is directed to a system for managing data privacy. The system can include a consent management server configured to receive, from a patient computing device, consent information including a first patient identifier, a plurality of health organization identifiers, and an indication that a patient corresponding to the first patient identifier consents to sharing of health information by a plurality of health organizations corresponding to the plurality of health organization identifiers. The consent management server can also be configured to reconcile the received consent information with stored consent information in a consent record in a database. The consent management server can also be configured to update, in the database, the consent record based on the reconciled consent information. The consent management server can also be configured to receive, from a data sharing organization, a query including a second patient identifier to determine whether the data sharing organization has permission to share health information related to the patient. The consent management server can be further configured to determine that the data sharing organization has permission to share health information related to the patient, based on the query and the consent record. The consent management server can be further configured to generate a response to the query based on the determination that the data sharing organization has permission to share health information related to the patient and to transmit the response to the data sharing organization.

In some implementations, the consent management server can be further configured to generate information for a graphical user interface comprising a plurality of interface elements including at least a first field corresponding to identification information for the patient and a second field corresponding to a health organization authorized by the patient to share health information related to the patient. The consent management server can also be configured to transmit the information for the graphical user interface to a computing device of the patient to cause the computing device of the patient to display the graphical user interface.

In some implementations, the consent management server can be further configured to receive, from the patient computing device, a response entered via the first field and the second field of the graphical user interface, the response corresponding to the consent information, and to update the consent record based on the response. In some implementations, the consent management server is further configured to generate information for a second graphical user interface comprising an interface element including at least a third field corresponding to a withdrawal of consent for the health organization to share health information related to the patient. The consent management server can also be configured to transmit the information for the second graphical user interface to the computing device of the patient to cause the computing device of the patient to display the second graphical user interface. In some implementations, the consent management server can be further configured to receive, from the patient computing device, a second response entered via the third field of the second graphical user interface. The consent management server can also be configured to update the consent record to indicate withdrawal of consent for the health organization to share health information related to the patient, based on the second response.

In some implementations, the consent management server can be further configured to determine that the data sharing organization has permission to share health information related to the patient by determining a first match between the first patient identifier and the second patient identifier and a second match between the data sharing organization and at least one of the plurality of health organization identifiers. In some implementations, the consent management server can be further configured to determine that the data sharing organization has permission to share health information related to the patient by determining that an expiration date of the consent record has not passed.

In some implementations, the consent management server can further be configured to reconcile the received consent information with the stored consent information by identifying a first portion of the received consent information that is redundant with respect to the stored information. The consent management server can discard the first portion of the received consent information, such that the reconciled consent information includes the stored consent information and a remaining portion of the received consent information that does not include the first portion of the received consent information.

In some implementations, the consent management server can further be configured to reconcile the received consent information with the stored consent information by identifying a first portion of the received consent information that is redundant with respect to the stored consent information and a remaining portion of the received consent information not including the first portion of the received consent information. The consent management server can discard the remaining portion of the received consent information, such that the reconciled consent information includes the first portion of the received consent information that is redundant with respect to the stored consent information.

Another aspect of this disclosure is directed to a method for managing data privacy. The method can include receiving, from a patient computing device, consent information including a first patient identifier, a plurality of health organization identifiers, and an indication that a patient corresponding to the first patient identifier consents to sharing of health information by a plurality of health organizations corresponding to the plurality of health organization identifiers. The method can include reconciling the received consent information with stored consent information in a consent record in a database. The method can include updating, in the database, the consent record based on the reconciled consent information. The method can include receiving, from a data sharing organization, a query including a second patient identifier to determine whether the data sharing organization has permission to share health information related to the patient. The method can include determining that the data sharing organization has permission to share health information related to the patient, based on the query and the consent record. The method can include generating a response to the query based on the determination that the data sharing organization has permission to share health information related to the patient. The method can include transmitting the response to the data sharing organization.

In some implementations, the method can include generating information for a graphical user interface comprising a plurality of interface elements including at least a first field corresponding to identification information for the patient and a second field corresponding to a health organization authorized by the patient to share health information related to the patient. In some implementations, the method can include transmitting the information for the graphical user interface to a computing device of the patient to cause the computing device of the patient to display the graphical user interface.

In some implementations, the method can include receiving, from the patient computing device, a response entered via the first field and the second field of the graphical user interface. The response can correspond to the consent information. In some implementations, the method can include updating the consent record based on the response. In some implementations, the method can include generating information for a second graphical user interface comprising an interface element including at least a third field corresponding to a withdrawal of consent for the health organization to share health information related to the patient. In some implementations, the method can include transmitting the information for the second graphical user interface to the computing device of the patient to cause the computing device of the patient to display the second graphical user interface. In some implementations, the method can include receiving, from the patient computing device, a second response entered via the third field the second graphical user interface. In some implementations, the method can include updating the consent record to indicate withdrawal of consent for the health organization to share health information related to the patient, based on the second response.

In some implementations, determining that the data sharing organization has permission to share health information related to the patient can include determining a first match between the first patient identifier and the second patient identifier and a second match between the data sharing organization and at least one of the plurality of health organization identifiers. In some implementations, determining that the data sharing organization has permission to share health information related to the patient can include determining that an expiration date of the consent record has not passed.

Another aspect of this disclosure is directed to a method for managing data privacy. The method can include generating a data structure storing a patient identifier corresponding to a patient and information associated with a plurality of attributes of the patient, including an identification of at least one healthcare provider associated with the patient. The method can include receiving information corresponding to a health event involving the patient. The method can include reconciling the received information with the data structure. The method can include updating the data structure to modify at least one of the plurality of attributes of the patient, based on the health event. The method can include determining whether the patient has provided consent for health information to be delivered to the at least one healthcare provider. The method can include determining a delivery method preference for the at least one healthcare provider, responsive to a determination that the patient has provided consent for the health information to be delivered to the at least one healthcare provider. The method can also include transmitting information corresponding to the patient identifier and the plurality of attributes to the at least one healthcare provider.

In some implementations, the method can include transmitting information corresponding to the health event to the at least one healthcare provider, responsive to the determination that the patient has provided consent for the health information to be delivered to the at least one healthcare provider. In some implementations, the method can include determining whether the patient has provided consent for a health information organization to receive and transport the health information, prior to transmitting the information corresponding to the patient identifier and the plurality of attributes to the at least one healthcare provider.

In some implementations, the method can include identifying a care team associated with the patient, the care team including a plurality of healthcare providers. The method can include identifying, for each of the plurality of healthcare providers included in the care team, a respective electronic address. The method can include transmitting, to each of the respective electronic addresses of the plurality of healthcare providers, a request for health information related to the patient.

In some implementations, the method can include receiving, from at least a subset of the plurality of healthcare providers, responses to the request for health information related to the patient. In some implementations, the method can include updating the data structure to modify at least one of the plurality of attributes of the patient, based on the responses. In some implementations, the method can include modifying the data structure to indicate that the patient has provided consent for the health information to be delivered to the at least one healthcare provider.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing.

FIG. 1A illustrates an example system for managing data privacy, according to an illustrative implementation.

FIG. 1B illustrates an example consent management system that can be included in the system of FIG. 1A, according to an illustrative implementation.

FIG. 1C illustrates an example active care relationship management system that can be included in the system of FIG. 1A, according to an illustrative implementation.

FIGS. 1D-1F illustrate example ledgers that can be used by components included in the system of FIG. 1A, according to illustrative implementations.

FIG. 2A illustrates a first example graphical user interface (GUI) that can be provided to a patient within the system of FIG. 1A, according to an illustrative implementation.

FIG. 2B illustrates a second example GUI that can be provided to a patient within the system of FIG. 1A, according to an illustrative implementation.

FIG. 2C illustrates a third example GUI that can be provided to a patient within the system of FIG. 1A, according to an illustrative implementation.

FIG. 2D illustrates a fourth example GUI that can be provided to a patient within the system of FIG. 1A, according to an illustrative implementation.

FIG. 3 illustrates a flowchart of a first example method for managing data privacy, according to an illustrative implementation.

FIG. 4 illustrates a flowchart of a second example method for managing data privacy, according to an illustrative implementation.

FIG. 5 illustrates an example system for collecting, maintaining, and updating patient information, according to an illustrative implementation.

FIG. 6 is a block diagram illustrating an exemplary computing device, according to an illustrative implementation.

FIG. 7 is a flow diagram illustrating a method for generating a common key for known persons, according to an illustrative implementation.

FIG. 8 is a flow diagram illustrating a method for generating a common key for unknown persons, according to an illustrative implementation.

FIG. 9 is a flow diagram illustrating a method for utilizing a known common key for a known person, according to an illustrative implementation.

FIG. 10 is a flow diagram illustrating a method for acquiring records using a common key, according to an illustrative implementation.

FIG. 11 is a flow diagram illustrating a method for verifying potential person matches, according to an illustrative implementation.

FIG. 12 is a flow diagram illustrating a method for updating files using a common key service, according to an illustrative implementation.

FIG. 13 is a flow diagram illustrating a method for utilizing a common key service in conjunction with a patient's hospital visit, according to an illustrative implementation.

FIG. 14 is a flow diagram illustrating a method for utilizing a common key service, according to an illustrative implementation.

FIG. 15 is a flow diagram illustrating a new patient's intake process, according to an illustrative implementation.

### DETAILED DESCRIPTION

Following below are more detailed descriptions of various concepts related to, and implementations of systems and methods for managing data privacy. The various concepts introduced above and discussed in greater detail below may be implemented in any of numerous ways, as the described concepts are not limited to any particular manner of implementation. Examples of specific implementations and applications are provided primarily for illustrative purposes.

In the healthcare field, a large volume of health information is stored electronically. New health information can be generated each time a patient experiences a health event resulting in contact with a health organization or healthcare provider. For example, each time a patient is admitted to a hospital, visits a physician's office, makes an insurance claim, or obtains medication from a pharmacy, new health information can be generated and stored to keep a record of the event. In some instances, such information may become part of the patient's electronic health record (EHR).

The information generated in this manner can be stored across a wide range of health systems. For example, information corresponding to a patient's hospital visit may be stored by a health information system maintained by the hospital. Similarly, information corresponding to the patient's insurance claim may be stored by a health information system maintained by the health insurance company that processes the claim, and information corresponding to the patient's prescribed medication may be stored by a health information system maintained by the pharmacy that fills the prescription. Thus, the medical information for a single patient may be distributed across a wide range of health information systems. In general, any health organization or healthcare provider, such as a hospital, a health plan, a medical insurer, a laboratory, a prescription benefit manager, a pharmacy, or a clinic, may maintain one or more health information systems each storing health information for any number of patients.

Often, the separate health information systems described above may not exchange information with one another on a regular basis. For example, while information for a given patient may be stored by both a hospital health information system and a medical insurer, neither the hospital nor the medical insurer may have a complete set of health information for the patient, because each may not have access to the information stored by the other. Moreover, federal and state regulations relating to patient privacy and protection of sensitive patient health information can make it difficult for organizations to safely and efficiently share patient health information with one another. For example, at least some health information, which can be referred to as specially protected information (SPI), may be subject to regulations requiring consent from a patient before the patient's SPI is transported between health organizations. Accurately obtaining and recording consent for a large population of patients can be difficult, as each patient may consent only to the sharing of certain types of SPI, or for only a particular subset of the health organizations that the patient is associated with to access the SPI. As a result, obtaining health information for a single patient can be difficult because it may not be clear where the desired information is stored or whether the patient has consented to the sharing of the information. This problem becomes more complex when trying to obtain health information for a population of multiple patients, all of whom may have health data stored by disparate health organizations and with different consent parameters.

In addition, new events involving the patient can require frequent updates to the patient's stored medical information. For example, as the patient visits new providers, is prescribed new treatments, or experiences new health events over time, information relating to these events should be recorded and made accessible to those involved in providing ongoing healthcare to the patient, to ensure that future medical treatment is consistent with the patient's full medical history. The patient may also update consent parameters associated with the patient's health information over time, for example by revoking consent for a particular health organization to receive or send specified types of SPI for the patient. In some instances, consent may also change over time without any action being taken by the patient, for example if the consent is subject to a validity period that can expire. Health organizations are cautious about transmitting patient SPI without knowing for certain that the patient has provided consent for them to do so. Thus, the difficulty in determining whether such consent exists can deter health organizations from sharing such information at all, even when the patient may have already provided consent to do so.

One technical problem presented in conventional computerized systems, such as the complex healthcare systems described above, is redundant storage of data elements. Computerized systems may store multiple data elements that are duplicates or may be different from each other but represent the same thing. For example, more than one data element may be stored to indicate that a patient gives consent to a particular healthcare provider because one data element has a different spelling of a name than another data element or another data element indicates a different healthcare provider at a common facility. Imperfect data can result from a variety of different errors such as spelling, incomplete names, abbreviations, and incomplete information. Multiple data elements that are identical or near-identical can result in redundant copies that take up data storage space and slow computational speeds.

The disclosed systems and methods can eliminate or reduce redundant storage of common elements including elements with imperfect data, which thereby improves efficiency, reduces electronic data storage costs, and provides better data integrity. The disclosed systems and methods also provide the advantage of better uniformity of data and, in the context of regulated data such as healthcare consent, the systems can improve adherence to federal and state laws, even when such laws change from time to time.

The disclosed systems and methods are technical solutions which can reconcile data elements to improve integrity and efficiency of the system. As such, the systems can be very efficient in responding to high volumes of queries. In a particular embodiment of a consent management system, the disclosed system and method capture the most appropriate consent information necessary while also centralizing and standardizing consent data for accuracy and efficient access. Such a system and method make the minimum necessary information available to organizations wanting data requiring consent.

This disclosure provides systems and methods for obtaining and recording patient health information and for obtaining and recording consent information related to the patient health information. An active care relationship management system can be used to store and update information relating to the healthcare providers or other health organizations currently associated with a patient. A consent management system can store information related to the entities the patient has authorized to transport or receive various types of health information. Health organizations and healthcare providers can query the active care relationship management system and the consent management system to determine where (i.e., at which other health organizations) health information is stored for a patient and whether they have permission to access or transmit the health information. Redundant health information can be eliminated, and data integrity can be improved. As a result, health organizations can more easily work together to share health information for a patient, subject to the patient's consent. The active care relationship management system and the consent management system can therefore help to resolve the challenges that result from the distributed storage of consent information and health information across a wide range of health information systems. These and other aspects of this disclosure are described further below.

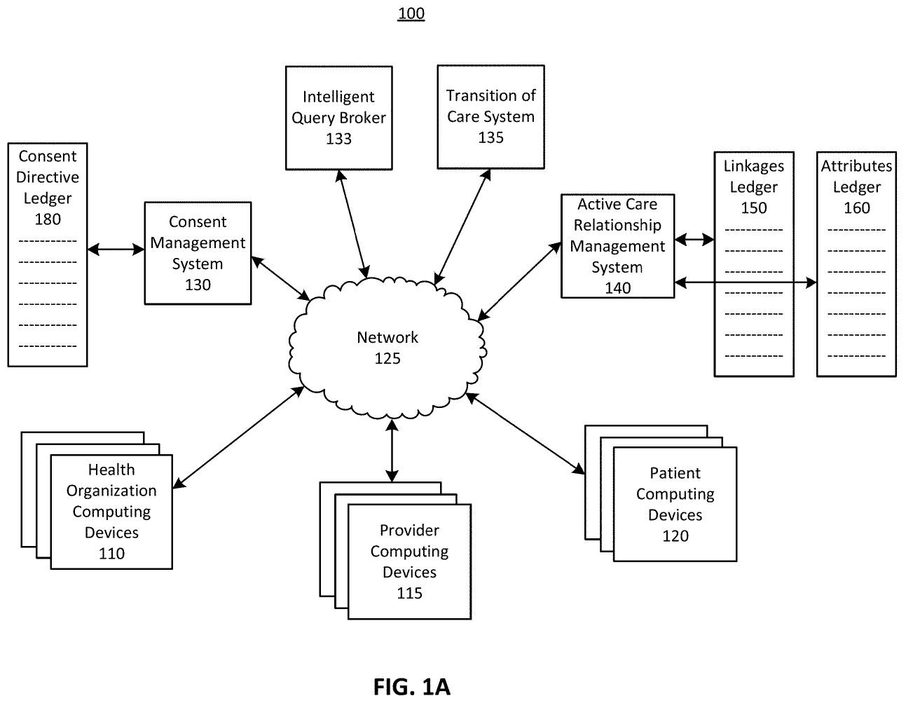

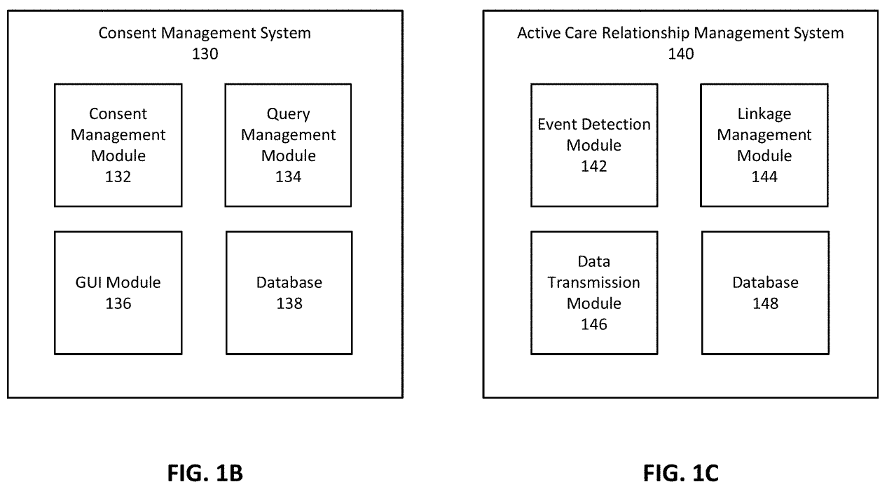

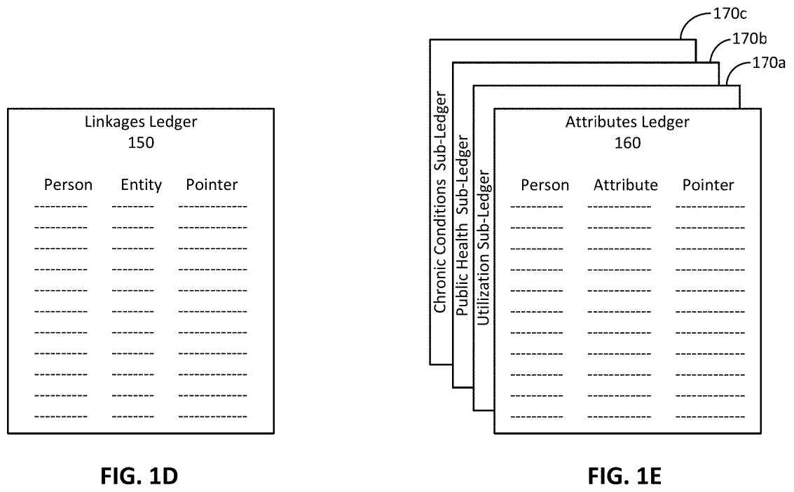

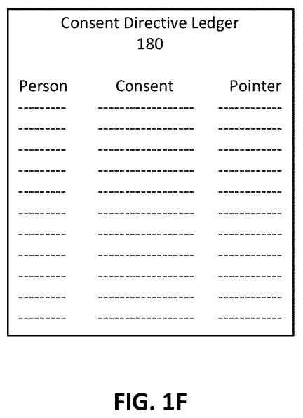

FIG. 1A illustrates an example system 100 for managing data privacy, according to an illustrative implementation. The system 100 includes a plurality of health organization computing devices 110, a plurality of provider computing devices 115, a plurality of patient computing devices 120, a consent management system 130, an intelligent query broker 133, a transition of care system 135, and an active care relationship management (ACRM) system 140, each of which is coupled to a network 125. In some implementations, the network 125 can include any of the Internet, a private network, a wide area network (WAN), or a combination thereof. It should be understood that, although only a single network 125 is shown in FIG. 1A for illustrative purposes, in some arrangements the components of the system 100 can be interconnected to one another via two or more interconnected computer networks that may be combined to implement the network 125.

FIG. 1B illustrates the consent management system 130, which includes a consent management module 132, a query management module 134, a GUI module 136, and a database 138. FIG. 1C illustrates the ACRM system 140, which includes an event detection module 142, a linkage management module 144, a data transmission module 146, and a database 148. FIGS. 1A-1C are described together below.

The ACRM system 140 is configured to assist with the collection and maintenance of patient health information, and the consent management system 130 is configured to ensure that patient health information is handled (e.g., transported, stored, etc.) in a manner consistent with the consent provided by the patients respectively associated with the health information. For example, the ACRM system 140 can receive medical information from the health organization computing devices 110, the provider computing devices 115, and the patient computing devices 120, and can process the received information to determine attributes of individual patients as well as relationships between various entities, including patients, providers (e.g., physicians, pharmacists, etc.), and other healthcare organizations.

In some implementations, the health organization computing devices 110 can be any type or form of computing device owned, operated, or otherwise accessed by a health organization, such as a health system, a hospital, a health plan, a medical insurer, a prescription benefit manager, a laboratory, a pharmacy, or a clinic. In some implementations, each of the health organization computing devices 110 can be implemented as at least one of a server, a desktop computer, or a laptop computer. In some other arrangements, each of the health organization computing devices 110 can be a mobile computing device such as a tablet computing device, or a handheld computing device, such as a smartphone that can be accessed by an employee or other representative of the respective health organization.

Similarly, the provider computing devices 115 and the patient computing devices 120 may also be any type or form of computing device owned, operated, or otherwise accessed by a healthcare provider or a patient, respectively. In general, a provider can include a physician, a pharmacist, a therapist, a laboratory technician, or any other person or group of people that provides healthcare to patients. In some arrangements, any of the provider computing devices 115 and the patient computing devices 120 can be implemented as at least one of a server, a desktop computer, a laptop computer, a tablet computing device, or a handheld computing device.

As described above, the ACRM system 140 can maintain health information for any number of patients, and may update the health information based on information received from the health organization computing devices 110, the provider computing devices 115, and the patient computing devices 120. For example, the ACRM system 140 can store information for patients in the form of data structures, such as the linkages ledger 150 and the attributes ledger 160. FIG. 1D shows an example implementation of the linkages ledger 150, and FIG. 1E shows an example implementation of the attributes ledger 160. The attributes ledger 160 includes a plurality of entries, each of which can be used to store information corresponding to a linkage between a person (e.g., a patient) and an entity (e.g., a health organization, a physician, etc.). In some implementations, the person field for an entry can be represented by a patient identifier for the patient. In some implementations, the attributes can include features such as medications prescribed to the patient (i.e., to the person corresponding to a given entry in the attributes ledger 160), diagnosed medical conditions of the patient, and demographic information for the patient. Entities represented in entries of the linkages ledger 150 can include healthcare providers with whom the patient has an active relationship (e.g., a primary care physician of the patient), health organizations that have treated the patient (e.g., a hospital to which the patient has been admitted), etc. For example, each entry in the linkages ledger 150 or the attributes ledger 160 can be or can include a data structure for a patient associated with one of the patient computing devices 120, and the data structure can include medical information relating to providers associated with any of the provider computing devices 115 or health organizations associated with any of the health organization computing device 110 with whom the patient has a medical relationship. In some implementations, each entry can be formatted as a data fractal. The data fractal can include three components, with each component corresponding to one of the fields of the entry (e.g., a person field, an entity field, and a pointer field for each entry in the linkages ledger 150). In some implementations, the linkage management module 144 can be configured to create and modify the linkages ledger 150 and the attributes ledger 160 for each respective patient, for example by adding new entries to the linkages ledger 150 and the attributes ledger 160 when appropriate. The linkages ledger 150 and the attributes ledger 160 can be stored, for example, in the database 148.

In some implementations, the pointer field for an entry can include a pointer to a particular endpoint in the system 100 (such as any of the health organization computing device 110, the provider computing devices 115, or the patient computing devices 120) where additional information related to the entry can be found. For example, a pointer can be or can include an electronic address of a computing device, such as a health organization computing device 110, a provider computing device 115, or a patient computing device 120, that may be queried to find additional information related to the information stored in that entry. The electronic address can be formatted, for example, as an email address or a URL. In some implementations, the pointer field for a single entry may include more than one pointer to a respective endpoint.

In some implementations, the event detection module 142 can parse information received from the health organization computing devices 110, the provider computing devices 115, and the patient computing devices 120, and can determine whether such information constitutes an event that should result in an update to the either the linkages ledger 150 or the attributes ledger 160. Such events can include any event that adds, removes, or alters a relationship between the patient and one or more health organizations or healthcare providers. For example, a provider could use a provider computing device 115 to transmit information to the ACRM system 140 indicating that the provider is treating a particular patient, and the event detection module 142 can determine that such information should be used to update the linkages ledger 150 to include a new entry to reflect the new provider-patient relationship. In another example, a health organization computing device 110 can transmit information to the ACRM system 140 corresponding to an admission, discharge, or transfer (ADT) event for a patient. The event detection module 142 can then determine that the event should result in an update to the data structure for the patient, and the linkage management module 144 can update the linkages ledger 150 accordingly (e.g., by storing information in the data structure indicating that the patient has been admitted, discharged, or transferred to the health organization). In another example, a health organization computing device 110 can transmit information to the ACRM system 140 corresponding to a new medical diagnosis for a patient. The event detection module 142 can then determine that the event should result in an update to the attributes ledger 160 to include a new entry to reflect the diagnosis for the patient.

In some implementations, the linkage management module 144 can be configured to reconcile information received by the ACRM system 140 when the event detection module 142 determines that the information corresponds to an event that should trigger an update to either the linkages ledger 150 or the attributes ledger 160. For example, reconciliation may include any operations performed by the linkage management module 144 in order to identify and/or correct the received information. Reconciliation may also include any operations performed by the linkage management module 144 to determine a match between a patient corresponding to the received health information and a patient whose information already is stored in one more existing entries of the linkages ledger 150 or the attributes ledger 160. Thus, by reconciling the received information with the existing information stored in the linkages ledger 150 and the attributes ledger 160, the linkage management module 144 can help to maintain data integrity and reduce data redundancy.

There may be inconsistencies in the format of the receive health information as compared to the format of information included in the entries of the linkages ledger 150 and the attributes ledger 160. For example, identifying information (e.g., a name stored in the “person” field of either the linkages ledger 150 or the attributes ledger 160 may include spaces, hyphens, periods, apostrophes, or other non-alphabet characters, while the received health information omits these characters. In another example, the linkages ledger 150 or the attributes ledger 160 may store information relating to a patient's gender as “male” or “female,” while the received health information formats the patient's gender as “M” or “F.” In some implementations, the received health information may include typographical errors, such as misspellings of words or transpositions of digits in numbers. These inconsistencies can make it difficult to determine an exact match between the received information and the information already stored in the linkages ledger 150 or the attributes ledger 160. The linkage management module 144 can be configured to reconcile the received health information with the existing information, for example, in order to avoid adding a new entry to the linkages ledger 150 or the attributes ledger 160 in instances in which an existing entry should be modified instead to reduce data redundancy.

In some implementations, the linkage management module 144 can use a common key service to facilitate data reconciliation. Each patient may be matched to a single identity, and that identity can be assigned a unique common key. The common key may be an alphanumeric sequence. The common key can be unique to each patient, and can be used to link records stored, housed, and/or generated by multiple health organizations and providers. For example, the “person” field of each entry in the linkages ledger 150 and the attributes ledger 160 can be or can include the common key for the patient to which the entry corresponds. By using a common key as a field of each entry, the linkage management module 144 can reduce the likelihood of complications and errors due to incomplete or incorrect information, and redundant data may be eliminated or reduced. Additional information relating to common key services similar to those that may be implemented by the linkage management module 144 to facilitate data reconciliation and to improve data integrity are described further below in connection with FIGS. 7-15.

In some implementations, the linkage management module 144 can reconcile newly received information with stored information by discarding redundant information. For example, the linkage management module 144 can determine an overlap between the received information and the stored information. To avoid redundancy, the linkage management module 144 can be configured to discard the overlapping portion of the received information, and may only store a remaining (i.e., non-overlapping) portion of the newly received information. Stated differently, newly received information can be considered a first set of information and stored information can be considered a second set of information, and the linkage management module 144 can be configured to store only the union of the first set of information and the second set of information. In some implementations, the linkage management module 144 can also delete previous entries in the linkages ledger 150 with which the newly received information is redundant, and can store information corresponding to the union in a new entry in the linkages ledger 150. Thus, the linkages ledger 150 may store a single entry corresponding to the union. In some other implementations, the linkage management module 144 can discard the overlapping portion of the newly received information and can add a new entry in the linkages ledger 150 corresponding to the remaining (i.e., non-overlapping) portion of the newly received information. Thus, the linkages ledger 150 may store information corresponding to the union across multiple entries that do not overlap with one another.

In some implementations, the linkage management module 144 can instead reconcile newly received information with stored information by preferring redundant information and discarding non-redundant information. The linkage management module 144 can determine that redundant information is likely to be more accurate. For example, the linkage management module 144 can determine an overlap between the received information and the stored information. The linkage management module 144 can be configured to discard the non-overlapping portion of the received information, as well as any stored information (e.g., entries in the linkages ledger 150) that are non-overlapping with respect to the newly received information. The linkage management module 144 can then record only the overlapping portion of the newly received information and the stored information. Stated differently, newly received information can be considered a first set of information and stored information can be considered a second set of information, and the linkage management module 144 can be configured to store only the intersection of the first set of information and the second set of information. In some implementations, the linkage management module 144 can delete previous entries in the linkages ledger 150 with which the newly received information is not redundant, such that only the overlapping information remains in the linkages ledger 150. It should be understood that the linkage management module 144 can also be configured to process newly received attribute information in a manner similar to that described above in connection with newly received linkage information. For example, the linkage management module 144 can be configured to add, delete, or modify entries in the attributes ledger 170 in order to save only a portion (e.g., a union or an intersection) of newly received attribute information and stored attribute information.

In some implementations, the linkage management module 144 can be configured to reconcile newly received information with stored information according to one or more policies. For example, the policies may be or may include policies to implement the union, intersection, or any other combination of newly received and stored information, as described above. In some other implementations, the policies may reconcile newly received information with stored information in a different manner. For example, newly received information may be reconciled with stored information on a field-by-field basis. In some implementations, a different policy may be applied for each field.

In some implementations, each of the linkages ledger 150 and the attributes ledger 160 can be implemented as blockchain of linked data structures each corresponding to an entry in its respective ledger. For example, each entry may include a pointer or other reference to the previous entry. In some implementations, such a pointer can be stored as a cryptographic hash. In some implementations, the linkages ledger 150 and the attributes ledger 160 can each represent a block in a blockchain whose accuracy is protected by one or more consensus techniques. For example, in some implementations, there may be multiple copies of each ledger stored at various locations accessible via the network 125. Addition of new entries to any of these copies, as well as modifications to old entries, may be protected by a proof-of-work mechanism that requires any entity adding or altering entries in the ledger to perform computational work before the addition or modification can be made. In some implementations, the entries themselves may include information relating to proof of the work that resulted in the creation of the entries. In some other implementations, the integrity of the ledgers may be protected by a proof-of-stake mechanism, rather than a proof-of-work mechanism.

Each entry in the linkages ledger 150 and the attributes ledger 160 also can include metadata, which may be any additional data relating to its corresponding entry. For example, in some implementations the metadata for an entry may include a timestamp corresponding to the time at which the entry was generated, or an identification of a data source (e.g., a health organization computing device 110) from which an event was received that triggered generation of the entry. Generally, the metadata for a field can be any type of data relating to that field.

In some implementations, there may be one or more sub-ledgers, such as the sub-ledgers 170a-170c (generally referred to as sub-ledgers 170) associated with the attributes ledger 160. For example, the sub-ledger 170a is a utilization sub-ledger that may store information relating to utilization of various medical resources by particular patients. For example, utilization may refer to actual use or predicted likelihood of future use of medical resources by a particular patient. In some implementations, utilization information may be binary. For example, utilization information may include an indication of whether or not a particular patient is classified as a “high-utilizer,” which may indicate that the patient has historically consumed a large share of medical resources. Utilization may be measured in terms of the monetary cost of treatment sought by a patient, a number of healthcare providers on a care team for the patient, durations of hospital stays for the patient, a number or frequency of visits to physicians, pharmacies, or other health organizations made by the patient, etc. The sub-ledger 170b is a public health sub-ledger. In some implementations, the public health sub-ledger can store information related to public health data. The sub-ledger 170c is a chronic conditions sub-ledger that may contain information relating to patients diagnosed with any of a variety of chronic medical conditions. In some implementations, the sub-ledgers 170 may store entries from which the entries stored in the attributes ledger 160 are derived or supplied. Thus, the sub-ledgers 170 can serve as an information source for the attributes ledger 160. In some other implementations, the attributes ledger 160 may include the sub-ledgers 170. For example, an entry in the attributes ledger 160 may be a sub-ledger 170. Thus, the attributes ledger 160 can be referred to as a “ledger of ledgers.” It should be understood that, in some implementations, there may be more or fewer sub-ledgers 170 than are illustrated in FIG. 1E. For example, there may be additional sub-ledgers 170 that may store information relating to medications or to social determinants of risk for various medical conditions. In some implementations, these ledgers may be maintained by other entities or may be generated based on information received by other entities, such as food banks, public health organizations, and the like.

The data transmission module 146 can be configured to transmit information to any of the health organization computing devices 110, the provider computing devices 115, or the patient computing devices 120. For example, the data transmission module 146 can transmit any information that may correspond to at least a portion of the information stored in an entry of the linkages ledger 150, the attributes ledger 160, or the sub-ledgers 170 for a patient, such as the patient identifier and information relating to one or more linked entities or attributes of the patient. In some implementations, the data transmission module 146 can transmit the information as any type of message or notification sent to the health organization computing devices 110, the provider computing device 115, or the patient computing devices 120, including an email, a text message, a phone call, or a mobile application notification. In some implementations, because data reconciliation can be performed as entries are added and/or modified in the linkages ledger 150 and the attributes ledger 160 to reduce data redundancy, the total volume of information transmitted by the data transmission module 146 may be reduced.

As described above, certain health information that may be stored in the database 148, such as any information characterized as SPI, may be subject to regulations that limit or restrict those who are permitted to access the information. For example, consent of the patient who is associated with the SPI (or a parent, legal guardian, or other agent of the patient) may be required before such information can be transmitted to or shared by a particular provider or health organization. In some implementations, the consent management system 130 can be configured to enable the health organization computing devices 110, the provider computing devices 115, the patient computing devices 120, and the ACRM system 140 to determine whether they are authorized to share or receive such information.

To accomplish this, the consent management system 130 can be configured to communicate with the patient computing devices 120 to allow users of the patient computing devices 120 (i.e., patients) to provide information related to their consent for certain health information to be shared. For example, the consent management module 132 can be configured to receive consent information from a patient computing device 120. The consent information can include an identifier of the patient associated with the patient computing device 120, such as a name, a portion of a social security number, demographic information, or any combination of these or other types of information that can be used to uniquely identify the patient from among a population of patients. The consent information can also include an indication that the patient consents to one or more health organizations or providers sharing the patient's health information. In some implementations, the consent information can also specify the type of health information subject to the consent. For example, the patient may wish to provide consent for behavioral health information to be shared, but may not consent to information relating to the patient's prescribed medications to be shared. In some implementations, the consent information can include information relating to a period of performance for which the patient is providing consent for the patient's health information to be shared. For example, the patient may specify that the consent is to expire on a particular date in the future or after a predetermined duration of time has elapsed.

The consent information can be formatted as stored as records in the consent directive ledger 180, which is depicted in FIG. 1F. Similar to the linkages ledger 150 and the attributes ledger 160, the consent directive ledger 180 can be a set of linked entries forming a blockchain. The blockchain can be protected by a proof-of-work mechanism or by a proof-of-stake mechanism, as described above. Each entry can include information corresponding to the person (e.g., a patient), consent information, and a pointer. For example, the consent information may be a specific the type of information that the patient consents to sharing, and the pointer field may indicate one or more entities who have the patient's consent to receive and/or transport the patient's health information. In some implementations, the consent information can be binary. For example, the consent information may indicate whether any type of consent is on file for the person, and there may be metadata for that entry that includes information relating to the particular consent stored for that person. In some implementations, the metadata can include a type of consent or an indication of one or more parties for whom the patient has provided consent to receive or transport health information. In some implementations, the metadata may also include additional information such as an expiration date for the consent. Each entry may be any type of data structure configured to store a record of the consent information. Thus, entries in the consent directive ledger 180 can be referred to as consent records. Generally, the entries of the consent directive ledger 180 can be assigned to or otherwise associated with the particular patient who provided the consent, which may correspond to the person field of that entry. In some implementations, the consent management module 132 can be configured to received consent information from a plurality of patients (e.g., via the patient computing devices 120) and a respective entry in the consent directive ledger 180 can be generated and assigned to each unique patient of the plurality of patients. The consent directive ledger 180 can be stored, for example, in the database 138.

In some implementations, the pointer for an entry in the consent directive ledger 180 can include an indication of an endpoint in the system 100 (such as any of the health organization computing device 110, the provider computing devices 115, or the patient computing devices 120) where additional information related to the entry can be found, similar to the pointers described above in connection with the linkages ledger 150 and the attributes ledger 160. For example, a pointer stored can be or can include an electronic address of a computing device, such as a health organization computing device 110, a provider computing device 115, or a patient computing device 120, that may be queried to find additional information related to the information stored in that entry. The electronic address can be formatted, for example, as an email address or a URL. In some implementations, the pointer field for a single entry may include more than one pointer to a respective endpoint.

In some implementations, the consent management module 132 can be configured to perform data reconciliation in order to improve the accuracy and integrity of the consent directive ledger 180, in a manner similar to that described above in connection with the linkage management module 144 of the ACRM system 140. For example, the consent management module 132 can be configured to identify inconsistencies in the formatting of received information as compared to the formatting of information stored in the consent directive ledger 180. The consent management module 132 can also be configured to determine when a mismatch occurs due to such an inconsistency that would not have occurred if the received data and the consent directive ledger 180 were formatted in a consistent fashion. For example, the consent management module 132 may implement a common key service in which each entry in the consent directive ledger 180 includes a field (e.g., the “person” field) that stores the common key for the patient to which the entry corresponds.

In some implementations, the consent management module 132 can also be configured to process newly received consent information in a manner similar to that described above in connection with newly received linkage information or newly received attribute information. For example, the consent management module 132 can be configured to add, delete, or modify entries in the consent directive ledger 180 in order to save only a portion (e.g., a union or an intersection) of newly received consent information and stored attribute information.

The information in the consent directive ledger 180 stored in the database 138 can be made available to data sharing organizations such as users of the health organization computing devices 110, the provider computing devices 115, or the patient computing devices 120 to allow the data sharing organizations to determine whether they are authorized to send or receive such information to other entities (e.g., other users of the health organization computing devices 110, the provider computing devices 115, or the patient computing devices 120). For example, data sharing organizations may submit electronic queries to the consent management system 130, which can be received by the query management module 134. A query may be formatted according to protocols such as Health Level 7 (HL7), Query By Parameter (QBP) as promoted by the Center for Disease Control, Fast Healthcare Interoperability Resources (FHIR), the Integrating the Healthcare Enterprise (IRE) framework, Cross Community Discovery (XDS), or any other suitable communications protocol.

A query can include a patient identifier for whom consent is sought. In some implementations, a query can also include an identifier of the data sharing organization (e.g., a health organization or provider) who is initiating the query, as well as information relating to the type or category of health information that the organization would like permission to share. In some implementations, a query can also include an identification of another entity with whom the data sharing organization wishes to share the patient's health information. For example, when a patient is admitted to a hospital, the hospital may request certain medical information from another entity such as a primary care physician of the patient, so that the hospital can understand the patient's medical history. In some implementations, the hospital may determine the identity of the patient's primary care physician by requesting this information from the ACRM system 140, as described above. Before transmitting any of the patient's health information, including SPI, to the hospital, the primary care physician may initiate a query (e.g., via a provider computing device 115) to the consent management system 130 to determine whether the patient has provided consent for the primary care physician to share the requested information with the hospital by referring to the consent directive ledger 180.

In some implementations, the query management module 134 can provide the query to the consent management module 132, which can determine whether the data sharing organization is authorized to share the health information specified in the query. For example, the consent management module 132 can be configured to make the determination by examining information stored in the entries of the consent directive ledger 180 that correspond to the patient whose information the data sharing organization wishes to share, as indicated by the patient identifier in the query. In some implementations, the consent management module 132 can determine whether the entries in the consent directive ledger 180 for the patient include information indicating that the patient has provided consent for the data sharing organization to share health information. For example, the consent management module 132 can make the determination by identifying a match between an identifier of a health organization or other entity for whom the patient has provided consent to data sharing as recorded in metadata of any of the entries in the consent directive ledger 180 corresponding to the patient, and an identifier of the data sharing organization who initiated the query. In some implementations, the consent management module 132 can also determine whether the patient's consent is still valid (i.e., that the consent has not been revoked by the patient or expired at the time the query is received). In some implementations, the consent management module 132 can also determine whether the patient's consent applies to the category or type of information specified in the query.

If the consent management module 132 determines that a valid consent exists for the data sharing organization (e.g., the consent directive ledger 180 indicates that the patient has authorized the data sharing organization to share the type of health information specified in the query, and the consent is still valid), the query management module 134 can generate a response to the query indicating that the data sharing organization is authorized to share the health information of the patient. Otherwise, if the consent directive ledger 180 indicates that the patient has not provided consent for the data sharing organization to share the requested information, or has not provided consent for the other entity specified in the query to receive the requested information, or if the patient previously provided consent for the requested information to be shared but the consent has since been revoked or become expired, the query management module 134 can generate a response to the query indicating that the data sharing organization is not authorized to share the health information of the patient.

As described above, the health organization computing devices 110, the provider computing devices 115, and the patient computing devices 120 may each interact with the consent management system 130 and the ACRM system 140 by sending electronic queries via the network 125. The consent management system 130 and the ACRM system 140 also may communicate with one another via the network 125. An electronic query may include any type or form of request for health information. Generally, a query may include one or more request parameters that specify the information requested by the query. For example, a request parameter may correspond to an entity (such as a healthcare provider, a patient, or a population of patients) for whom health information is requested. A request parameter also may specify an attribute such as a quality measure, a characteristic, or a value identifying the type of information requested. For example, a request parameter may indicate a health condition to which the query pertains.

Queries may be formatted according to a variety of protocols, such as Health Level 7 (HL7), Query By Parameter (QBP) as promoted by the Center for Disease Control, Fast Healthcare Interoperability Resources (FHIR), the Integrating the Healthcare Enterprise (IRE) framework, Cross Community Discovery (XDS), or any other suitable communications protocol. In some implementations, each component of the system 100 may be configured to submit only a subset of available protocols, which may be different from the subset of protocols supported by the other components of the system 100.

To facilitate communication of queries and results between the components of the system 100, in some implementations these queries may be handled by the intelligent query broker 133. For example, the intelligent query broker 133 can be configured to intercept queries in the system 100. The intelligent query broker 133 can be configured to determine one or more destination endpoints, such as the health organization computing devices 110, the provider computing devices 115, the patient computing devices 120, the consent management system 130, the transition of care system 135, or the ACRM system 140 that may store the information requested by the query. In some implementations, the intelligent query broker 133 can also adaptively translate the query into various formats associated with the respective destination endpoints, forward the translated queries to the destination endpoints, and receive responses to the queries from the destination endpoints. The intelligent query broker 133 can then return any information received in the responses back to the source endpoint.

In some implementations, the transition of care system 135 can be configured to use information provided by the consent management system 130 and the ACRM system 140 to facilitate changes to the care team of a patient. For example, when responsibility for an aspect of a patient's medical care is transferred from one health organization or physician to another, or when a patient establishes a relationship for a new aspect of the patient's medical care, the transition of care system 135 can be configured to retrieve the patient's relevant medical information from the ACRM system 140 and provide it to the new health organization or provider.

In some implementations, the transition of care system 135 can query the ACRM system 140 to retrieve information corresponding to the patient's past medical care, which may be stored in the linkages ledger 150 or the attributes ledger 160 as described above. In addition, to ensure that the new health organization or provider is authorized to receive such information, the transition of care system 135 can query the consent management system 130 to determine whether the patient's consent for transportation of such information is valid. If the patient's consent is not valid, the transition of care system 135 can prompt the consent management system 130 to attempt to obtain the consent, as described further below. In some implementations, the ACRM system 140 can predict that a healthcare organization or provider is likely to require a patient's consent in the near future. For example, the linkages ledger 150 or the attributes ledger 160 may store information indicating that a new provider has recently been added to the patient's care team. As a result, the new provider may not yet have received consent to access the patient's health information. To address this, the ACRM system 140 can transmit a query to the consent management system 130 to determine whether the new provider has consent to access the patient's health information. If the provider does not have consent, the consent management system 130 can be configured to prompt the patient to provide consent for the new provider to access the patient's health information, for example by proving one or more GUIs to a patient computing device 120 to allow the patient to electronically provide the required consent, as described further below. Thus, the prompt for the patient's consent can be “pushed” to the patient before either the patient or the new provider affirmatively requests the consent. This can help to save time and improve the patient's experience of transitioning to a new healthcare provider.

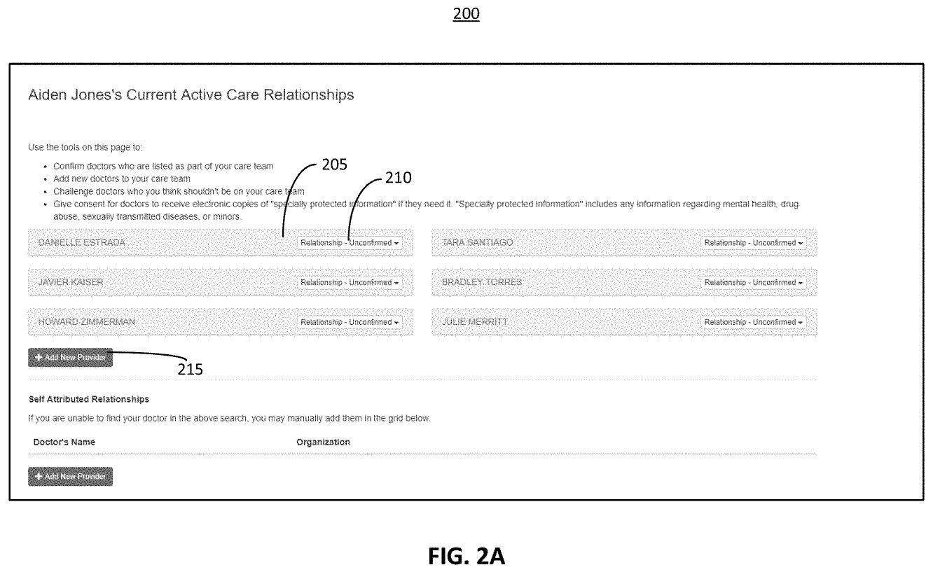

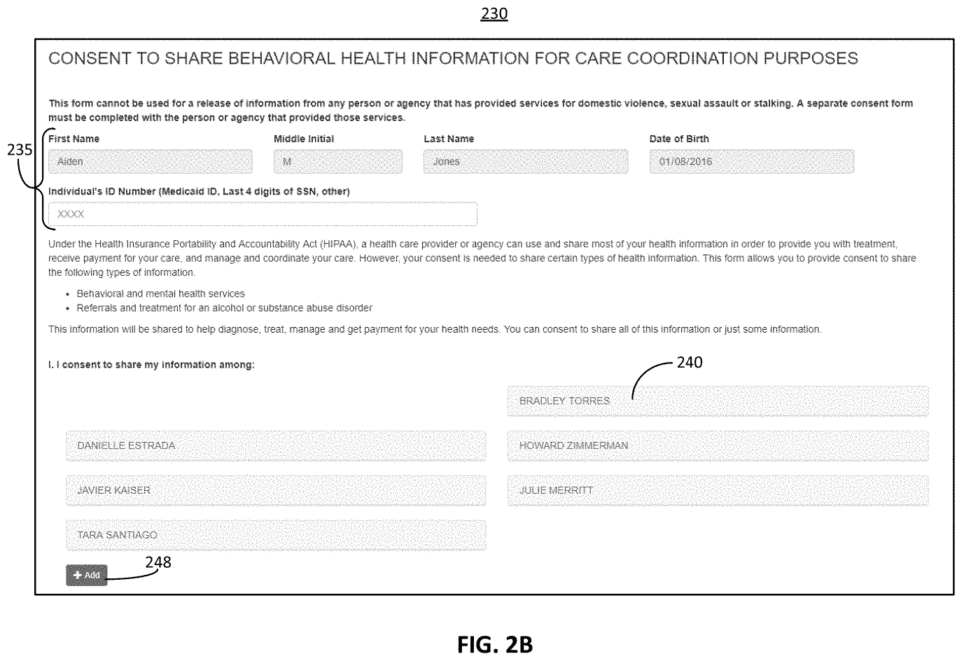

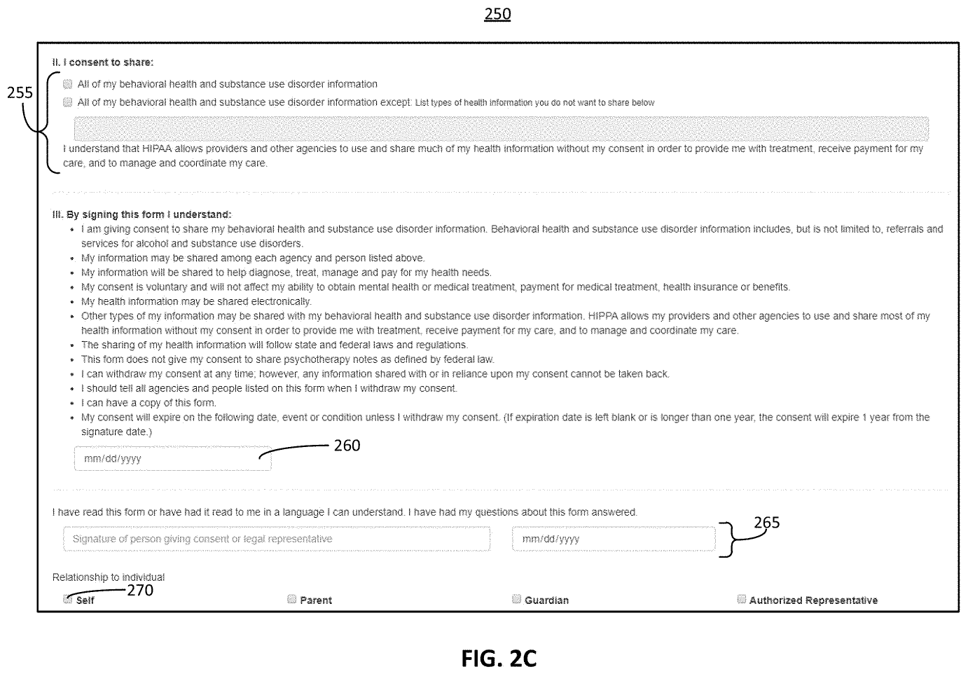

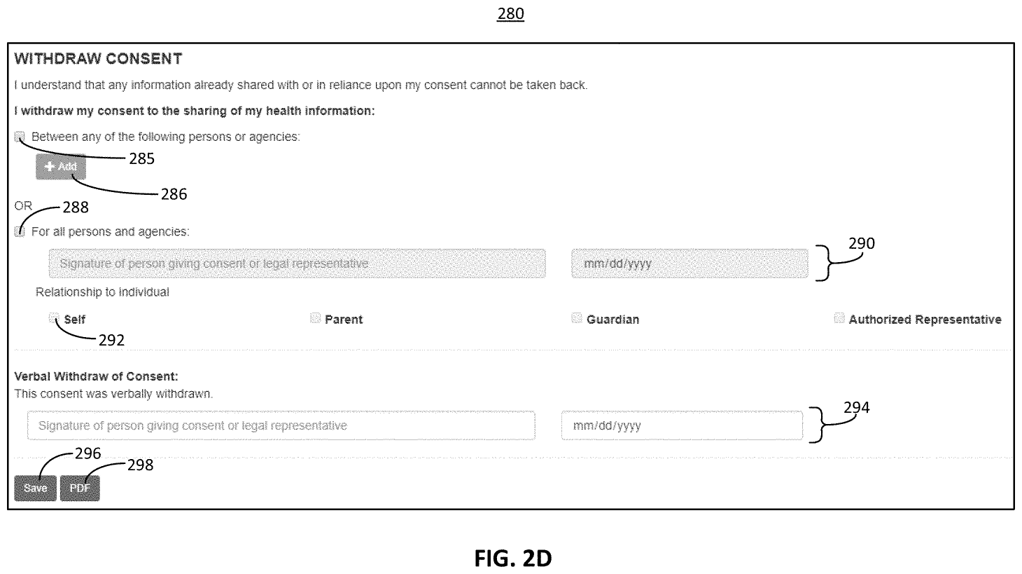

Referring again to FIG. 1B, in some implementations, the GUI module 136 of the consent management system 130 can be configured to generate and provide one or more GUIs that can enable a patient to provide consent for the sharing of the patient's health information. For example, such a GUI can be used to allow the patient to provide the consent information used by the consent management module 132 to generate or modify the consent record for the patient. In some implementations, the GUI module 136 can generate information for such a GUI and can transmit the information to a patient computing device 120 in a format that allows the GUI to be rendered via a display device or other output device of the patient computing device 120. The patient can then interact with the patient computing device 120, for example via one or more input devices included in or otherwise accessible by the patient computing device 120, to provide the consent information via the GUI. The consent information can then be transmitted from the patient computing device 120 to the consent management module 132, which can use the consent information to generate or update the consent record for the patient. FIGS. 2A-2D show example GUIs that can be generated by the GUI module 136.

FIG. 2A illustrates a first example GUI 200 that can be provided to a patient within the system 100 of FIG. 1A, according to an illustrative implementation. For example, information corresponding to the GUI 200 can be generated by the GUI module 136 and transmitted to a patient computing device 120 to cause the patient computing device 120 to render the GUI 200 to a user of the patient computing device 120. In some implementations, the 200 can allow the user (e.g., a patient) to provide information corresponding to active healthcare relationships that the patient has with any number of providers or other healthcare organizations. As shown, the GUI 200 includes text notifying the user that the user can use the GUI to add or confirm healthcare providers who are part of the user's care team, and to challenge providers who the user believes should not be part of the user's care team. The GUI 200 includes a plurality of fields such as the field 205, each of which indicates the name of a provider that may be part of the user's care team. In some implementations, the fields 205 may be pre-populated (e.g., populated without any input from the user). For example, the fields 205 may be populated to include any or all of the healthcare organizations or providers that are linked to the user within the linkages ledger 150. For each named provider, the GUI 200 includes a respective dropdown menu such as the dropdown menu 210. The dropdown menu 210 can be used to indicate whether the user wishes to confirm or dispute that the respective provider as part of the user's care team. The GUI also includes a button 215 that can be selected to add a new provider who is not currently listed in the GUI 205. For example, if the user recently began seeing a new healthcare provider who is not yet listed as a part of the user's care team, the user may use the button 215 to add the new healthcare provider to the user's care team.

In some implementations, the user can interact with the dropdown menu 210 and the button 215 using any suitable interface device, such as a mouse, a trackball, a stylus, a touchscreen, a keyboard, or any other type of input device that can allow the user to interact with the dropdown menu 210 or the button 215. In some implementations, the GUI can be configured to capture input selections made by the user through the interface elements of the GUI including the dropdown menu 210 and the button 215, and to transmit information corresponding to the user's input selections to the consent management system 130 or the ACRM system 140 via the network 125. For example, information corresponding to providers that the user confirms or disputes can be transmitted to the ACRM system 140 in order to allow the ACRM system 140 to update the data structure to indicate the confirmed or disputed relationship between the patient and the selected providers, as described above.

FIG. 2B illustrates a second example GUI 230 that can be provided to a patient within the system of FIG. 1A, according to an illustrative implementation. In some implementations, the GUI 230 can allow the user to provide consent for health information relating to behavioral and mental health services, as well as referrals and treatment for alcohol or substance abuse disorder, with one or more providers or healthcare organizations. The GUI 230 includes a plurality of fields 235 corresponding to identification information for the user. In some implementations, the user can enter the user's identification information, for example using a keyboard or other user input device, and the GUI 230 can be configured to transmit the information to the consent management system 130 via the network 125. In some implementations, at least a portion of the user's identification information may be pre-populated in the GUI 230 before the user provides any input. For example, the identification information may be retrieved from any of the linkages ledger 150, the attributes ledger 160, or the consent directive ledger 180, and used to pre-populated the GUI 230 when the GUI 230 is first displayed to the user. The GUI 230 also includes a plurality of fields 240 each corresponding to a respective provider or healthcare organization to whom the consent applies. In some implementations, the user can use a pointing device or other user input device to remove or modify providers shown in these fields 240. The user can also select the button 248 to add a new provider or health organization not already displayed in the GUI 230.

After the user has made selections through the GUI 230, the third example GUI 250 shown in FIG. 2C can be displayed to the user. The GUI 250 includes a plurality of user interface elements 255, which can include checkboxes and text fields, that allow a user to select a variety of types of information for which the user's consent applies. The GUI 250 also includes text notifying the user of the consequences of providing consent, as well as signature fields 265 and an optional expiration date field 260. The GUI 250 also includes a plurality of relationship fields 270 asking the user to confirm the user's relationship to the patient for whom consent is being provided. In some implementations, after the user has made selections via the GUIs 230 and 250, information corresponding to the selections can be transmitted to the consent management system 130 via the network 125. In some implementations, the consent management system 130 can use the information to generate or modify a consent record for the user, which can be stored in the database 138.

FIG. 2D illustrates a fourth example GUI 280 that can be provided to a patient within the system of FIG. 1A, according to an illustrative implementation. In some implementations, the GUI 280 can be provided to a user via a patient computing device 120 to allow the user to withdraw consent for the user's health information to be shared by a provider or health organization to whom the user had previously provided consent. The GUI 280 provides a checkbox 285 that allows the user to specify the particular providers or health organizations for whom the user wishes to revoke consent for data sharing. For example, after selecting the checkbox 285, the user can use the button 286 to add the providers or health organizations for whom the user wishes to revoke consent for data sharing. Alternatively, the user can select the checkbox 288 to withdraw consent all providers and health organizations to share the user's health information, without any need to list the providers or health organizations for whom the consent is to be revoked. The GUI 290 also provides signature fields 290, relationship checkboxes 292 for the user to indicate the user's relationship to the patient for whom consent is being revoked. If consent was withdrawn verbally, such as during a medical procedure undergone by the patient, the patient can confirm this withdrawal via the verbal withdrawal of consent fields 294, which require a signature and date.

The GUI 280 also includes a save button 296 that can allow the user to save a record of the information entered via the GUI 280. In some implementations, selecting the save button 296 can cause the information to be saved locally on the patient computing device 120. In some implementations, selecting the save button 296 can cause the information to be transmitted to the consent management system 130, to allow the consent record for the patient to be updated accordingly. The GUI 280 also includes a PDF button 298, which the user can select to view a copy of the information entered into the GUI 280 in portable document format (.pdf) format.

The GUIs 200, 230, 250, and 280 shown in FIGS. 2A-2D can be displayed on the patient computing device 120 in any suitable format. For example, in some implementations the GUIs 200, 230, 250, and 280 can be displayed as part of a mobile application executing on the patient computing device 120. In some implementations, the GUIs 200, 230, 250, and 280 can be rendered within a web browser application executing on the patient computing device 120. For example, the user of the patient computing device 120 can cause the patient computing device 120 to render the GUIs 200, 230, 250, and 280 within the web browser application by accessing a uniform resource locator (URL) of a website hosted by the consent management system 130, which in turn can cause the GUI module 136 to generate and transmit information corresponding to the GUIs 200, 230, 250, and 280 to the patient computing device 120.

In some implementations, the consent management system 130 can provide the GUIs 200, 230, 250, and 280 to the patient computing device 120 in response to determining that a provider or health organization has been denied permission to share or access the patient's health information. For example, the consent management system 130 can receive a query from a health organization computing device 110 or a provider computing device 115, and the determine that the patient has not provided consent for the health organization or provider associated with the query to share or access the patient's health information. In response, the GUI module 136 can generate and transmit information corresponding to the GUIs 200, 230, 250, and 280 to the patient computing device 120 associated with the patient, in order to allow the patient to update the consent to include the health organization or provider associated with the query, if the user so desires.

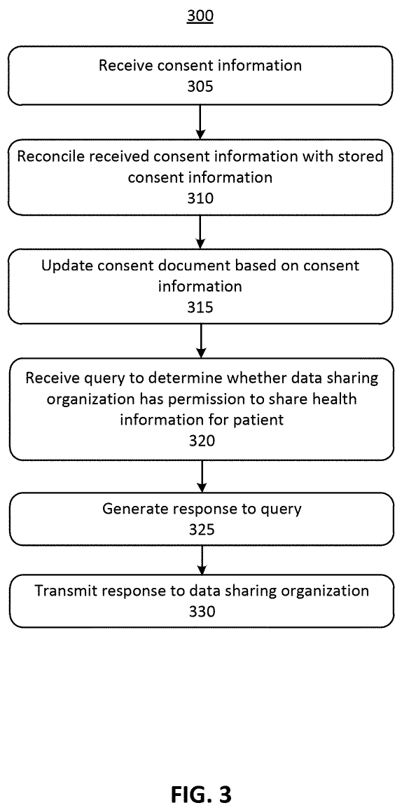

FIG. 3 illustrates a flowchart of a first example method 300 for managing data privacy, according to an illustrative implementation. In some implementations, the method 300 can be performed by the consent management system 130 shown in FIGS. 1A and 1B. In brief overview, the method 300 includes receiving consent information (operation 305), reconciling the received consent information with stored consent information (operation 310), updating a consent record based on the reconciled consent information (operation 315), receiving a query to determine whether a data sharing organization has permission to share health information for a patient (operation 320), generating a response to the query (operation 325), and transmitting the response to the data sharing organization (operation 330).

Referring again to FIG. 3, the method 300 includes receiving consent information (operation 305). In some implementations, the consent information can be received from a patient computing device such as the patient computing devices 120. of FIG. 1A. For example, the consent information can be collected based on user inputs via a GUI displayed on the patient computing device 120. The consent information can include a first patient identifier, which can uniquely identify the patient for whom the consent information is supplied. For example, the consent information can include the patient's name, social security number, other identification number, or any combination of these or other types of identifying information. In some implementations, the consent information can also include an indication that the patient consents to the sharing of health information with at least one health organization. For example, the consent information can also include a health organization identifier uniquely identifying a health organization for whom the patient is providing consent. In some implementations, the consent information can also include a provider identifier for a healthcare provider, such as a physician, for whom the patient is providing consent.

The method 300 includes reconciling the received consent information with stored consent information (operation 310). In some implementations, the consent management module 132 can perform the reconciliation based on the received consent information and one or more consent records stored in a database. In some implementations, the consent record can be any type or form of data structure, including a data fractal. For example, the consent record may be at least a portion of an entry stored in a ledger such as the consent directive ledger 180. In some implementations, the consent management module 132 may store a set of policies corresponding to common data formatting inconsistencies, as well as policies for addressing or correcting the inconsistencies. For example, the policies may include one or more rules or steps to be performed to convert data included in the received consent information into a format that is consistent with the formatting of the stored consent information (e.g., the format used for entries in the consent directive ledger 180). In some implementations, reconciling the received consent information with the stored consent information can include identifying redundant data (e.g., the same data included in both the received consent information and the stored consent information) and discarding the redundant data. In some implementations, the consent management module 132 can use a common key service to perform at least a portion of the reconciliation.

The method 300 includes updating the consent record based on the consent information (operation 315). In some implementations, the consent management module 132 can update the consent record based on the consent information received in operation 305. In some implementations, the consent management module 132 can generate a new consent record corresponding to the received consent information. For example, the consent management module 132 can generate the consent record in the form of any type of data structure configured to store the consent information. In some implementations, the consent record can be formatted as an extensible markup language (XML) document. The consent record can be assigned to or otherwise associated with the particular patient who provided the consent information. In some implementations, if a consent record already exists for the patient, the consent management module 132 can instead modify the existing consent record based on the consent information received in operation 305, rather than generating a new consent record. The consent management module 132 can store the consent record, for example, in the database 138. For example, the consent record can be stored as an entry in the consent directive ledger 180, or as a portion of an entry in the consent directive ledger 180.

The method 300 includes receiving a query to determine whether a data sharing organization has permission to share health information for a patient (operation 320). The query can be received, for example, by the query management module 134 from a health organization computing device 110 or a provider computing device 115. The query can include a second patient identifier corresponding to the patient for whom consent to share health information is sought. In addition, the query may include an identifier of a health organization or provider with whom the health organization initiating the query would like to share the patient's health information. For example, the health organization that initiates the query may wish to share the patient's health information with another health organization of provider, but may be required to obtain the patient's consent before sharing the health information.

The method 300 includes generating a response to the query (operation 325). In some implementations, the consent management module 132 can first determine whether the data sharing organization has permission to share the patient's health information, based on the query and the patient's stored consent record. For example, the consent management module 132 can make the determination by identifying a match between the second patient identifier included in the query and the first patient identifier associated with the consent information received in operation 305. This match can allow the consent management module 132 to locate the consent record for the appropriate patient. The consent management module 132 can also identify a match between the first identifier of a health organization or other entity for whom the patient has provided consent to data sharing as recorded in the patient's consent record, and the identifier of the data sharing organization that initiated the query. In some implementations, the consent management module 132 can also determine whether the patient's consent is still valid (i.e., that the consent has not been revoked by the patient or expired at the time the query is received). In some implementations, the consent management module 132 can also determine whether the patient's consent applies to the category or type of information specified in the query.

If the consent management module 132 determines that a valid consent exists for the data sharing organization, the query management module 134 can generate a response to the query indicating that the data sharing organization is authorized to share the health information of the patient. Otherwise, if the consent management module 132 determines that a valid consent does not exist for the data sharing organization, the query management module 134 can generate a response to the query indicating that the data sharing organization is not authorized to share the health information of the patient. The method 300 also includes transmitting the response to the data sharing organization (operation 330). In some implementations, the response can be transmitted via the network 125.

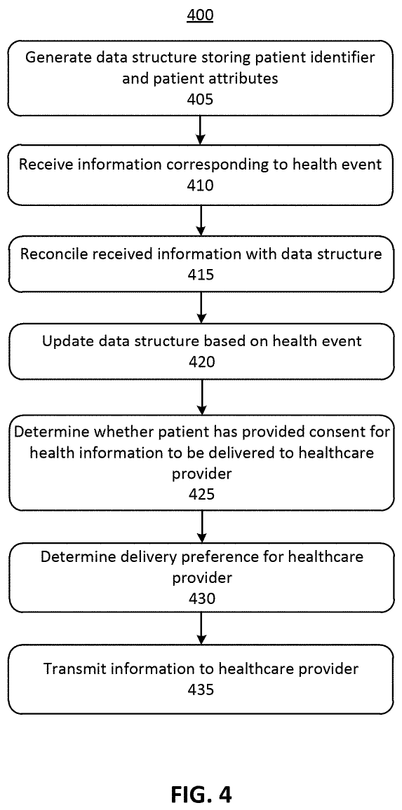

FIG. 4 illustrates a flowchart of a second example method 400 for managing data privacy, according to an illustrative implementation. In some implementations, the method 400 can be performed by the ACRM system 140, or by the consent management system 130, or by both the ACRM system 140 and the consent management system 130. In brief overview, the method 400 includes generating a data structure storing a patient identifier and a plurality of attributes (operation 405), receiving information corresponding to a health event involving the patient (operation 410), reconciling the received information with the data structure (operation 415), updating the data structure based on the health event (operation 420), determining whether the patient has provided consent for health information to be delivered to at least one healthcare provider included in the data structure (operation 425), determining a delivery preference for the healthcare provider (operation 430), and transmitting information to the healthcare provider (operation 435).

Referring again to FIG. 4, the method 400 includes generating a data structure storing a patient identifier and a plurality of attributes (operation 405). In some implementations, this operation can be performed by the linkage management module 144 of the ACRM system 140 shown in FIG. 1C. The patient identifier can correspond to a patient, and may include any type or form of information uniquely identifying the patient from among a plurality of patients. For example, in some implementations the patient identifier can be a common key identifier for the patient. In some implementations, the attributes can include at least one healthcare provider associated with the patient. For example, the healthcare provider can be any provider who has examined, treated, or otherwise interacted with the patient. In some implementations, the healthcare provider can be a member of a care team that treats the patient, which may include any number of other healthcare providers or health organizations. The attributes can also include other health information for the patient, such as medications that have been prescribed to the patient or any of the patient's diagnosed medical conditions. The linkage management module 144 can generate the data structure to be stored in a in a larger data structure, such as the attributes ledger 160. For example, the data structure may correspond to an entry or a portion of an entry in the attributes ledger 160. In some implementations, the data structure can be a data fractal.

The method 400 includes receiving information corresponding to a health event involving the patient (operation 410). In some implementations, this operation can be performed by the event detection module 142 of the ACRM system 140. For example, the event detection module 142 can receive information from any of the health organization computing devices 110, the provider computing devices 115, or the patient computing devices 120 shown in FIG. 1A via the network 125. The event detection module 142 can parse the information received from the health organization computing devices 110, the provider computing devices 115, and the patient computing devices 120, and can determine whether such information constitutes an event that should result in a change to the data structure associated with the patient. Such events include any event that adds, removes, or alters a relationship between the patient and one or more healthcare providers or health organizations. For example, a provider could use a provider computing device 115 to transmit information to the ACRM system 140 indicating that the provider is treating a particular patient, and the event detection module 142 can determine that such information should be used to update the data structure for the patient to reflect the new provider-patient relationship.

The method 400 includes reconciling the received information with the data structure (operation 415). In some implementations, the linkage management module 144 can perform the reconciliation based on the received information and any portion of the data structure generated in operation 405. In some implementations, the linkage management module 144 may store a set of policies corresponding to common data formatting inconsistencies, as well as policies for addressing or correcting the inconsistencies. For example, the policies may include one or more rules or steps to be performed to convert data included in the received information into a format that is consistent with the formatting of the data structure (e.g., the format used for entries in the consent directive ledger 180). In some implementations, reconciling the received information with the data structure can include identifying redundant data (e.g., the same data included in both the received information and the data structure) and discarding the redundant data. In some implementations, the linkage management module 144 can use a common key service to perform at least a portion of the reconciliation.

The method 400 includes updating the data structure based on the health event (operation 420). In some implementations, this operation can be performed by the linkage management module 144 of the ACRM system 140. For example, if the event of operation 410 corresponds to an ADT event for the patient to a particular health organization, the linkage management module 144 can update the data structure by storing information in the data structure indicating that the patient has been admitted, discharged, or transferred to the health organization.

The method 400 includes determining whether the patient has provided consent for health information to be delivered to at least one healthcare provider included in the data structure (operation 425). In some implementations, this operation can be performed by the ACRM system 140. For example, the data structure for the patient can include information corresponding to active patient consent for each healthcare provider stored in the data structure for the patient, and the linkage management module 144 can determine whether the patient has provided consent for a particular health provider to receive the patient's health information. In some implementations, the ACRM system 140 can instead query the consent management system 130 to determine whether the patient has provided consent for health information to be delivered to the at least one healthcare provider included in the data structure. For example, the data transmission module 146 can transmit information corresponding to a query to the query management module 134 of the consent management system 130, and the consent management system 130 can respond to the ACRM system 140 in a manner similar to that described above in connection with operations 320, 325, and 330 of the method 300 shown in FIG. 3. The response can indicate whether the patient has provided consent for health information to be delivered to the at least one healthcare provider.

If an active consent exists for the at least one healthcare provider, the method 400 can include determining a delivery preference for the healthcare provider (operation 430). In some implementations, the delivery preference can include an electronic address at which the healthcare provider prefers to receive healthcare information for the patient. For example, the electronic address can include an email address or a URL for the healthcare provider. In some implementations, the delivery preference can include a file format in which the healthcare provider prefers to receive the health information, or a communications protocol in which the healthcare provider prefers to receive the health information. The method 400 also includes transmitting information to the healthcare provider (operation 435). This operation can be performed, for example, by the data transmission module 146 of the ACRM system 140. In some implementations, the information can include the patient identifier of the patient, as well as the plurality of attributes for the patient, that are recorded in the data structure for the patient. The data transmission module 146 can format and transmit the information in accordance with the delivery preference.

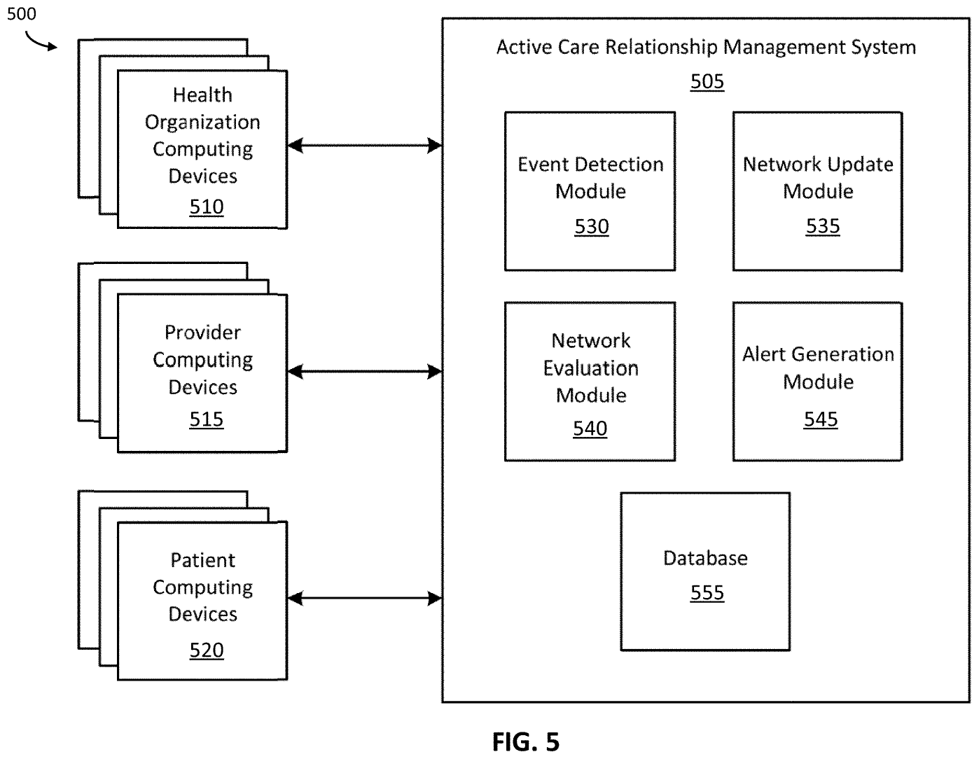

Referring to FIG. 5, there is shown an exemplary system 500 for collecting, maintaining, and updating patient information in accordance with an embodiment of the present disclosure. The system 500 includes an active care relationship management (ACRM) system 505 that may be used to provide an active care relationship service (ACRS), a plurality of health organization computing devices 510, a plurality of provider computing devices 515, and a plurality of patient computing devices 520. The ACRM system 505 includes an event detection module 530, a network update module 535, a network evaluation module 540, an alert generation module 545, and a database 555.

The ACRM system 505 is configured to assist with the collection, maintenance, and distribution of patient information. For example, the ACRM system 505 may receive patient information from the health organization computing devices 510, the provider computing devices 515, and the patient computing devices 520, and may process the received information to determine relationships between various entities, including patients, physicians, and other healthcare providers. The ACRM system 505 may generate and continuously update a semantic network based on the information received from the health organization computing devices 510, the provider computing devices 515, and the patient computing devices 520. The semantic network may be an interconnection of nodes, which may include patients, medications, medical conditions, providers, hospitals, clinics, etc., as described in U.S. patent application Ser. No. 15/847,506, which is incorporated herein in its entirety.

In some embodiments, the generation and update of the semantic network may be carried out by the event detection module 530 and the network update module 535. For example, the event detection module 530 may parse information received from the health organization computing devices 510, the provider computing devices 515, and the patient computing devices 520, and determine whether such information constitutes an event necessitating an update to the semantic network. The network update module 535 may add a node corresponding to either a provider or a patient to the semantic network if such nodes are not already present in the semantic network, and may add or modify the interconnection among the nodes. The network update module 535 also may store semantic information for the node interconnection. The network evaluation module 540 may analyze the semantic network to make inferences that may be relevant to the healthcare of one or more patients in the network. For example, the network evaluation module 540 may be configured to recognize patterns that may be used to predict healthcare outcomes or to select preventive care strategies for patients. The alert generation module 545 may be configured to provide alerts, such as an email, a text message, a phone call, or a mobile application notification, to any of the health organization computing devices 510, the provider computing devices 515, or the patient computing devices 520. The database 555 may store information about the semantic network, consent rules, policies, etc.

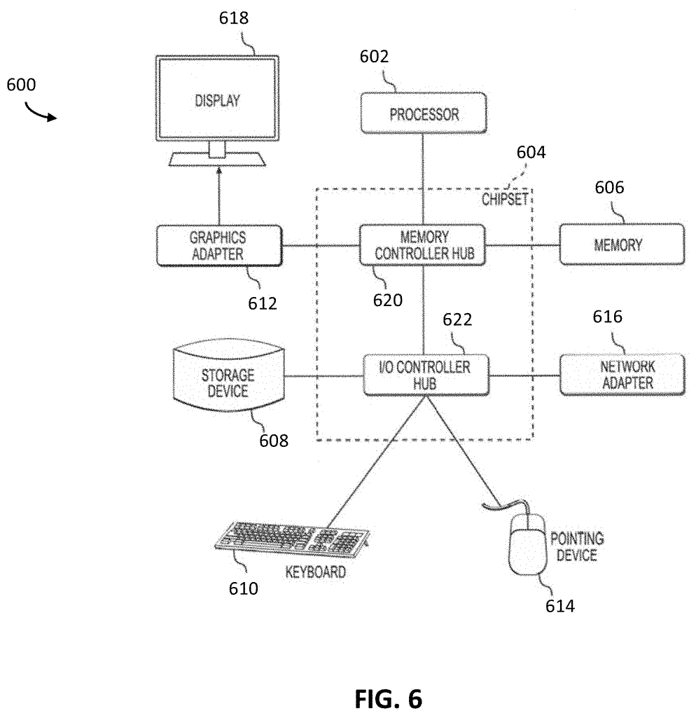

In some embodiments, the health organization computing devices 510 may be any type or form of computing device owned, operated, or otherwise accessed by a health organization, such as a health system, a hospital, a health plan, a medical insurer, a prescription benefit manager, a pharmacy, or a clinic. In some arrangements, each of the health organization computing devices 510 is implemented as at least one of a server, a desktop computer, or a laptop computer. An exemplary computing device will be described below with respect to FIG. 6. In some other arrangements, each of the health organization computing devices 510 may be a mobile computing device such as a tablet computing device, or a handheld computing device, such as a smartphone that may be accessed by an employee or other representative of the respective health organization.

Similarly, the provider computing devices 515, the patient computing devices 520, and the ACRM system 505 may also be any type or form of computing device owned, operated, or otherwise accessed by a provider, a patient, and a ACRS provider, respectively. In general, a provider may include a physician, a pharmacist, or any other person or group of people that provides healthcare to patients. In some embodiments, the provider computing devices 515, the patient computing devices 520, or the ACRM system 505 may be implemented as at least one of a server, a desktop computer, a laptop computer, a tablet computing device, or a handheld computing device.

FIG. 6 is a block diagram illustrating an exemplary computing device 600 in accordance with an embodiment of the present disclosure. In alternative embodiments, fewer, additional, and/or different components may be present. The computing device 600 may be any computing machine, such as a tablet, smart phone, laptop computer, desktop computer, server, remote client device, gaming device, smart television device, wearable computer, or any combination thereof. The computing device 600 may include at least one processor 602 coupled to a chipset 604. The chipset 604 may include a memory controller hub 620 and an input/output (I/O) controller hub 622. A memory 606 and a graphics adapter 612 may be coupled to the memory controller hub 620, and a display 618 may be coupled to the graphics adapter 612. A storage device 608, a keyboard 610, a pointing device 614, and a network adapter 616 may be coupled to the I/O controller hub 622. Other embodiments of the computing device 600 may have different architectures.

The storage device 608 may be a non-transitory computer-readable storage medium such as a hard drive, compact disk read-only memory (CD-ROM), DVD, or a solid-state memory device (e.g., read only memory (ROM) and random access memory (RAM)). The memory 606 may hold instructions and data that may be used by the processor 602. The pointing device 614 may be a mouse, a track ball, or other type of pointing device, and may be used in combination with the keyboard 610 to input data into the computing device 600. The pointing device 614 may also be a gaming system controller, or any type of device used to control a gaming system. For example, the pointing device 614 may be connected to a video or image capturing device that employs biometric scanning to detect a specific user. The specific user may employ motion or gestures to command the point device 614 to control various aspects of the computing device 600. The graphics adapter 612 may display images and other information on the display 618. To enhance interaction with a user, the herein disclosed embodiments may be implemented using an interactive display, such as a graphical user interface (GUI). Such GUIs may include interactive features such as pop-up or pull-down menus or lists, selection tabs, and other features that may receive human inputs. The network adapter 616 may couple the computer system device 600 to one or more computer networks.

The computing device 600 may be adapted to execute one or more computer programs for providing functionality described herein. A computer program (also known as a program, module, engine, software, software application, script, or code) may be written in any form of programming language, including compiled or interpreted languages, declarative or procedural languages, and the program may be deployed in any form, including as a stand-alone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program may be deployed to be executed on one computing device 600 or on multiple computing devices 600 that may be located at one site or distributed across multiple sites and interconnected by a communication network. In one embodiment, program modules may be stored into the storage device 608, loaded into the memory 606, and executed by the processor 602.

As used herein, the term module refers to computer program logic used to provide the specified functionality. Thus, a module may be implemented in hardware, firmware, and/or software. As used herein, the term processor encompasses all kinds of apparatus, devices, and machines for processing data, including by way of example a programmable processor, a computer, a system on a chip, or multiple ones, or combinations, of the foregoing. The processor may include special purpose logic circuitry, e.g., an FPGA (field programmable gate array) or an ASIC (application-specific integrated circuit). The processor also may include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

The types of computing devices used by the entities and processes disclosed herein may vary depending upon the embodiment and the processing power required by the entity. The computing device 600 may be a mobile device, tablet, smartphone or any sort of computing element with the above-listed elements. For example, a data storage device, such as a hard disk, solid state memory or storage device, may be stored in a distributed database system comprising multiple blade servers working together to provide the functionality described herein. The computer devices may lack some of the components described above, such as keyboards 610, graphics adapters 612, and displays 618.

As would be appreciated by one of ordinary skill in the art, the embodiments disclosed herein may be implemented on any system, network architecture, configuration, device, machine, or apparatus, and is not to be construed as being limited to any specific configuration, network, or systems, even though an example system is shown and described with respect to FIG. 6. The embodiments herein may be suitably implemented on conventional computing devices, for example, computer workstations, on Internet based applications, on optical computing devices, neural computers, biological computers, molecular computing devices, and other devices. As may be appreciated by those skilled in the art, the present invention, in short, may be implemented on any system, automaton, and/or Turing machine.

An automaton is herein described as a mechanism that is relatively self-operating and designed to follow a predetermined sequence of operations or respond to encoded instructions. A Turing machine is herein described as an abstract expression of a computing device that may be realized or implemented on an infinite number of different physical computing devices. Examples of systems, automatons and/or Turing machines that may be utilized in performing the process of the present invention include, but are not limited to: electrical computers (for example, an International Business Machines (IBM) personal computer); neuro-computers (for example, one similar to the “General Purpose Neural Computer” described in U.S. Pat. No. 5,155,802, issued to Paul H. Mueller, on Oct. 13, 1992); molecular computers (for example, one similar to the “Molecular Automata Utilizing Single or Double-Strand Oligonucleotides” described in U.S. Pat. No. 5,804,373, issued to Allan Lee Schweiter et al., on Sep. 8, 1998); biological computers (for example, one similar to the biological computer presented by Ehud Shapiro, of the Computer Science and Applied Mathematics Department at the Weizman Institute of Science (Rehovot, Israel), at the Fifth International Meeting on DNA-Based Computers); and optical computers. For purposes of simplicity, such devices hereinafter are referred to as computers, as is commonly understood in the art. But, the embodiments disclosed herein are not limited being applied to such devices and may be accomplished upon any system or collection of systems capable of providing the features and functions identified herein.

Multiple computing devices 600 may be clustered to form a computing system of clients and servers. A client and server are generally remote from each other and typically interact through a communications network. The relationship of client and server arises by virtue of computer programs running on the respective computing devices and having a client-server relationship to each other. In some embodiments, a server transmits data (e.g., an HTML page) to a client (e.g., for purposes of displaying data to and receiving user input from a user interacting with the client device). Data generated at the client (e.g., a result of the user interaction) may be received from the client at the server.

The methods of generating and utilizing common keys in a common key service, described below with respect to FIGS. 7-15, may be performed partially or wholly on a processor, such as the one described above with regards to the computing device 600. A common key provides a way to match patients and their records across multiple organizations, applications, and services, such as an ACRS supported by an ACRM system (e.g., the ACRM system 505). With a common key, full and complete records for a patient may be accessed or modified as desired. Even if multiple records are associated with a patient, a common key may link the various records together. A common key service is safe and secure, protects confidentiality, and has high accuracy and data integrity of records shared across the multiple entities.

In an embodiment of the present disclosure, exchange of information between health organizations and providers may be performed utilizing common key services. Each patient may be matched to a single identity, and that identity is assigned a unique common key. The common key may be an alphanumeric sequence. The common key is unique to each patient, and is used to link records stored, housed, and/or generated by multiple health organizations and providers.

The common key, when associated with the single identity of a patient, may be used to associate records of the patient across multiple entities and data stores across the healthcare market, such as primary care physician records, specialist records, hospital records, demographic records, billing information records, insurance information records, medical history records, care coordinator records, research databases, oncological databases, behavioral health system databases, pharmacy databases, etc.

A common key service may be used to link patients to their respective electronic medical records. Records may come from various formats and be stored across disparate systems. A common key may be used to link various records of a patient together. Additionally, a patient's demographic information (e.g., address, name, billing information, etc.) may change over time, making demographic information outdated or subject to error. By using a common key to keep track of records, complications and errors in treating patients due to incomplete information and records may be reduced.

A common key service may minimize mismatched record/patient associations and ensure that the record keeping is accurate. Matches and links between a patient and the patient's records may be affected across multiple organizations, applications, and services. This may lead to higher data integrity, which in turn improves patient safety by minimizing mistakes made by those utilizing the data. For example, a common key service may provide enhanced patient safety and care coordination by ensuring that medication and allergy information is tied to the correct person through a continuum of care, enhancing patient safety and potentially reducing the risk of medical errors. A common key service may also reduce work completed to coordinate care to patients across different organizations, applications, and services. This may also reduce cost of record keeping in service industries, such as health care. Furthermore, a common key service may be implemented to utilize current health information exchanges (HIE) and healthcare information technology (HIT) systems.

In an embodiment of the present disclosure, an HIT or HIE endpoint may be mapped to a master person index (MPI) using common key services. For example, a governmental body such as a state government may maintain an MPI. The MPI may contain a record of every person in the state or known to the state. Such an MPI may be populated using information acquired through different government entities, such as entities that issue identification, process taxes, process health benefits, or schools. The common key service may insure that any medical records are mapped to a single identity of an individual as maintained on the MPI. In one embodiment of the present disclosure, the systems and methods disclosed herein for a common key service may be executed as a web service that utilizes application programming interface (API) calls to the MPI and/or HITs and HIEs. Such an embodiment may allow for easy integration of an MPI, HIT, and/or HIE with the common key service.

In one embodiment of the present disclosure, the common key service may be used in a case that tracks an active care relationship of a provider or providers with a patient in the healthcare industry. For example, a Patient X is admitted to a hospital. The hospital may be a part of a data sharing organization (DSO). Such a DSO may include other health care providers or hospitals that are a part of a single health system. A provider, such as a physician, at the hospital may generate an admit-discharge-transfer (ADT) notification that indicates Patient X has been admitted to the hospital. The ADT notification may be sent to an ACRS supported by an ACRM system (e.g., ACRM system 505) via the DSO that invokes the common key service. In other words, when the DSO attempts to store the ADT notification, the ACRS (which may be offered separately from or in conjunction with the common key service) will invoke or reference a common key from the common key service to determine and/or verify that the ADT notification is properly associated with Patient X and stored properly.

The common key service then utilizes demographic information received in conjunction with the ADT notification to search an MPI for the identity of Patient X. The MPI may be maintained by a state agency, as indicated above, or may be stored and maintained by another entity, such as the entity offering the common key service. If a match for Patient X is found in the MPI and Patient X is already associated with a common key, the MPI sends back to the common key service Patient X's unique common key. In this way, the ADT notification may be properly recorded and associated with the true identity of Patient X in the active care relationship files. Similarly, the common key may also be used by the DSO and the ACRS to determine other records relating to Patient X, which may facilitate proper care to Patient X while Patient X is treated at the hospital.

If the MPI finds a true identity of Patient X based on the demographic information, but Patient X is not yet associated with a unique common key, the MPI will request a common key from the common key service. A common key is generated by the common key service and that common key is assigned to Patient X. Similar to the above, the common key may then be added to the appropriate records. If the MPI does not find an identity match for Patient X or a common key, the MPI may create a new person record and request a new unique common key from the common key service. Examples of persons that may not have a record or common key may be a newborn or a person that has no previous affiliation with the body maintaining the MPI.

In another example, Patient X may be admitted to a hospital and the DSO associated with the hospital may already be aware of a common key associated with Patient X. Accordingly, when an ADT notification is generated, the DSO may send the ADT notification to the ACRS as well as the common key service along with the known common key. Accordingly, the records may be stored and associated with the common key, and the common key service may not have to look up the common key and match an identity in the MPI. However, in an alternative embodiment, the common key service may still look up the common key and the identity of Patient X in the MPI so as to verify that all of the information from the DSO is correct.

In another embodiment of the present disclosure, the common key service may be utilized to anonymize and make records more secure and private. For example, after a common key has been established for a patient and known by DSOs, health care providers, an MPI, the common key service, etc., personal identifying information about the patient may no longer be transmitted between those various entities. For example, a patient's records may be updated using only the patient's common key to identify which records to update, and the patient's name, birthdate, social security number, address, other demographic data, etc. may not be used to update the patient's medical records. In this way, the transmission of the patient's medical records and the medical records themselves may be de-identified.

In another embodiment of the present disclosure, a different use case may be applied in conjunction with a common key service. In the embodiment, a researcher may be studying a medical condition and utilizing records to perform a study. For example, the researcher may be studying the effectiveness of depression treatment as a cancer patient progresses through chemotherapy. Different treatments for a cancer patient may be administered by multiple providers for a single patient. Records for the different treatment may be generated and/or stored on multiple different systems. Accordingly, locating accurate information across disparate systems for a single patient may be difficult. For example, the name John Smith is very common, and if he is a subject of the study, linking together different medical records with various medical records numbers may be difficult. In other words, the researcher may find it difficult to piece together exactly which John Smith got what treatments, when the treatments were administered, and where the treatments were administered because, in part, each system may utilize different medical record numbers for the various John Smiths that exist. Accordingly, the researcher may utilize the common key system to determine which of the various records relating to a John Smith related to the John Smith that is the subject of the study. In this way, the research may be more easily and accurately conducted. In one embodiment, the records may not be associated with a common key, so the common key service matches each record to a true identity associated with the John Smith that is the subject to the study. In another embodiment, the researcher may find records that are already associated with a common key. In this instance, the researcher may utilize the common key service to request the common key of the John Smith that is the subject of the study. Once the common key for the correct John Smith is determined, the records that include the same common key may be easily identified, either by the researcher or automatically by the common key service. In other words, this embodiment allows researchers to link information from various systems to the appropriate patients using the common key services.

The common key services systems and methods disclosed herein overcomes difficulties in matching patients to facilitate the exchange of health information, despite medical information being stored in disparate systems. For example, one hospital registration/admission system may record gender as Male, Female, Unknown, while another hospital system may list M, F, or U instead. Furthermore, inconsistencies in patient demographics may also complicate accurate matching. For example, a patient's name may be entered as Jane Smith-Jones in one system; Jane Smith Jones (without the hyphenation (-)) in another system; and a third system may record her name as Jane Jones. In one system, Jane's address may be her most recent, while another system still has her address as her previous home; one may have her date of birth with year 1975 while the others have her birth year as 1957. In an embodiment of the present disclosure, when a provider or DSO requests records for a particular patient (or records associated with a particular common key), the system may format records according to the way the requesting party stores and transmits patient data and records. In this way, a requesting party may more easily interpret, display, and store the requested information according to the requesting party's system.

To streamline the exchange of information to support meaningful use and accountable care, electronic health care systems utilize reliable matching using a common key service as disclosed herein to determine that the right information is attributed to the right patient every time. The common key services disclosed herein provide a consistent and reliable way to match patients across multiple organizations, applications and services. Such reliable matching capabilities ensure patient safety and high data integrity when data is shared. The common key service links information for individuals or organizations by using best practices for matching criteria to ensure that identifiers and attributes positively and accurately identify multiple types of entities. Examples of attributes that may be used to match identities include demographic information such as name, date of birth, gender, etc. In an alternative embodiment, information unique to the patient such as biometric information (fingerprint, palm scan, eye scan, etc.) may be used to determine an identity match. Individuals and entities that may use a common key service may include patients, beneficiaries, physicians and physician organizations, payers and health plans, hospitals and health systems, health care facilities, public health entities, etc. The common key services disclosed herein may allow accurate data sharing through a wide variety of use cases, such as results delivery, hospital notifications, public health reporting, care coordination and patient safety, quality and administrative reporting, patient engagement, infrastructure (e.g. ACRS, statewide health provider directory, information security/identity management), standard consent, quality assurance systems, etc.

The common key service disclosed herein provides means to link information for individuals or organizations accurately in support of various use cases. For example, improved patient matching increases the volume of outbound ADT messages that accurately reach providers and payers, which helps a widespread health system operate more efficiently. The common key systems and methods disclosed herein may also leverage a state's MPI which may utilize robust processes for managing information about persons and de-duplicating entries with great accuracy.

In an embodiment of the present disclosure, the common key service receives a request for a patient's common key and passes the request to a respective state's MPI. Such a request may result in the following actions: (1) If the patient is found in the MPI, then the common key service creates a common key for the patient and cross-references it with the state's MPI to ensure accurate mapping across systems. (2) If a person is not found in the state's MPI, then the common key service assigns a common key and passes it to the state's MPI, which creates or modifies a record for that patient in the MPI. (3) If a potential match or possible duplicate is identified in the state's MPI the requestor receives a list of possible matches and is prompted to review the records in detail to identify the correct patient and/or to identify errors that caused the duplication in the MPI (e.g., misspelled name, incorrect birth date). The requestor then sends a message to the common key service which informs the MPI as to which of the duplicates is the right person. If the MPI is the source for the duplicate data, an MPI staff may review the data and correct duplicates and errors. Such a process may help ensure that person records are kept up-to-date, improving the integrity of the common key service and making both the MPI and common key service more robust. A record may be modified or created in the MPI by sending a message from the common key service that includes an action and any associated common keys. For example, the action in a message may be one or more of merge, update, or delete. Common keys to merge, update, or delete would be included in the message and associated with the appropriate action in the message.

Additionally, the common key service may utilize an ACRS supported by an ACRM system (e.g., ACRM system 505). An ACRM system or similar records management system may be exposed to the common key service via an API and then mapped to and integrated with the MPI using its APIs. This may yield an ease of technical implementation. For example, a Medicaid population that is serviced using an ACRS may be easily applied and used with a common key service.

FIG. 7 is a flow diagram illustrating a method 700 for generating a common key for known persons in accordance with an embodiment of the present disclosure. In alternative embodiments, fewer, additional, and/or different operations may be performed. Also, the use of a flow diagram is not meant to be limiting with respect to the order of operations performed. In an operation 705, a common key service receives a request for an individual's common key. In an operation 710, the common key service sends a request to an MPI. In an alternative embodiment, the MPI may instead be any database that stores information on persons and de-duplicates records on individual persons. In an operation 715, the individual is found in the MPI, but the individual is not associated with a common key. In an operation 720, the common key service generates a common key. In an operation 725, the common key service sends the common key to the MPI, such that the common key may be associated with the record of the individual in the MPI. In an alternative embodiment, the MPI and the common key service may not be separate systems. In other words, the common key system may store persons similar to an MPI, may determine the person matches in the MPI according to requests from data sharing organization devices or providers, and may use the common keys to de-duplicate records regarding single individuals. In other words, the common key system and the MPI may be the same or multiple systems that achieve the same functions as disclosed herein.

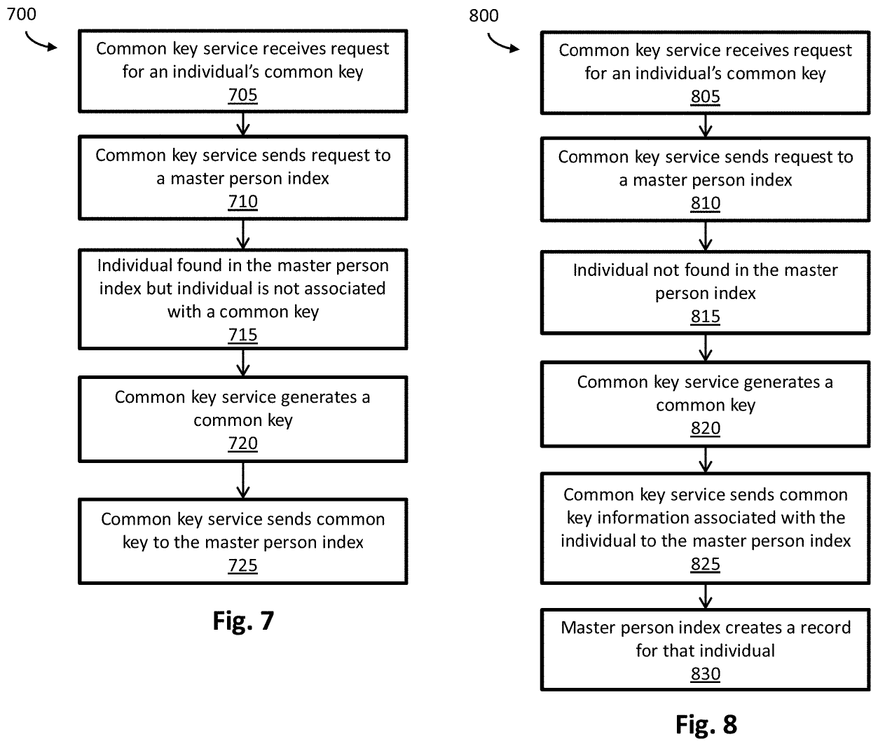

FIG. 8 is a flow diagram illustrating a method 800 for generating a common key for unknown persons in accordance with an embodiment of the present disclosure. In alternative embodiments, fewer, additional, and/or different operations may be performed. Also, the use of a flow diagram is not meant to be limiting with respect to the order of operations performed. In an operation 805, a common key service receives a request for an individual's common key. In an operation 810, the common key service sends a request to an MPI. In an operation 815, the individual is not found in the MPI. In an operation 820, the common key service generates a common key for the individual in response to a record of the individual not being found in the MPI. In an operation 825, the common key service sends the generated common key information associated with the individual to the MPI. In an operation 830, the MPI creates a record for that individual that includes identifying demographic information and the common key.

FIG. 9 is a flow diagram illustrating a method 900 for utilizing a known common key for a known person in accordance with an embodiment of the present disclosure. In alternative embodiments, fewer, additional, and/or different operations may be performed. Also, the use of a flow diagram is not meant to be limiting with respect to the order of operations performed. In an operation 905, a common key service receives a request for an individual's common key. In an operation 910, the common key service sends a request to an MPI. In an operation 915, the individual and an associated common key are found in the MPI. In an operation 920, the common key service sends the common key received from the MPI to the requestor device. Additionally, in another embodiment, the common key service may also associate a record from the requestor with the common key.

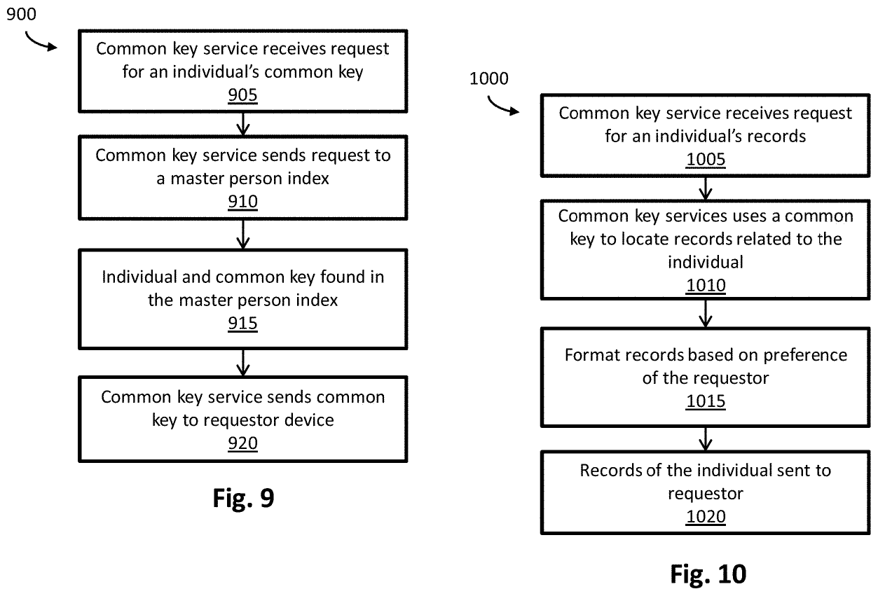

FIG. 10 is a flow diagram illustrating a method 1000 for acquiring records using a common key in accordance with an embodiment of the present disclosure. In alternative embodiments, fewer, additional, and/or different operations may be performed. Also, the use of a flow diagram is not meant to be limiting with respect to the order of operations performed. In an operation 1005, the common key service receives a request for an individual's records. In an operation 1010, the common key service uses a common key to locate records related to the individual. In one embodiment, the records located may be stored in the common key system. In another embodiment, the records may be located on other systems, such as a DSO system, an ACRM system, a provider system, an MPI, or other locations where records may be stored. The common key utilized to locate or identify the records may be determined in various ways such as the methods described above with respect to FIGS. 7-9. In an operation 1015, the records located are formatted based on a format preference of the requestor. This formatting may affect the way certain information/data is presented, and may affect what information/data is presented. In other words, the system may format the delivered records form and determine which records or portions of records should actually be delivered. In an operation 1020, the records located and formatted are sent to the requestor.

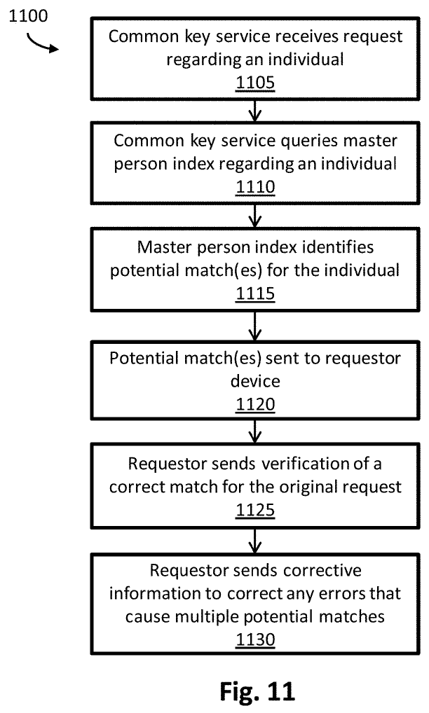

FIG. 11 is a flow diagram illustrating a method 1100 for verifying potential person matches in accordance with an embodiment of the present disclosure. In alternative embodiments, fewer, additional, and/or different operations may be performed. Also, the use of a flow diagram is not meant to be limiting with respect to the order of operations performed. In an operation 1105, the common key service receives a request regarding an individual. In an operation 1110, the common key service queries an MPI regarding the individual. In an operation 1115, the MPI identifies potential matches (or a single potential match) for the individual. In an operation 1120, the potential match(es) are sent to the requestor device. In an operation 1125, the requestor sends verification of a correct match for the original request to the common key service and/or the MPI. In other words, the requestor confirms which of the potential match(es) is the correct match. In an operation 1130, the requestor may also send corrective information to correct any errors that cause multiple matches. For example, some demographic information of an individual may be stored incorrectly in the MPI such that the MPI erroneously returned that individual as a potential match. The request may, in the operation 1130, correct the error to prevent future mistakes and make the MPI and the common key service more accurate and helpful. In an alternative embodiment, confirmation of a correct match and/or corrective information regarding a record may be sent from an entity or device other than the requestor. For example, the common key service or the MPI may also be able to determine a correct match from potential match(es) and correct incorrect information in a record.

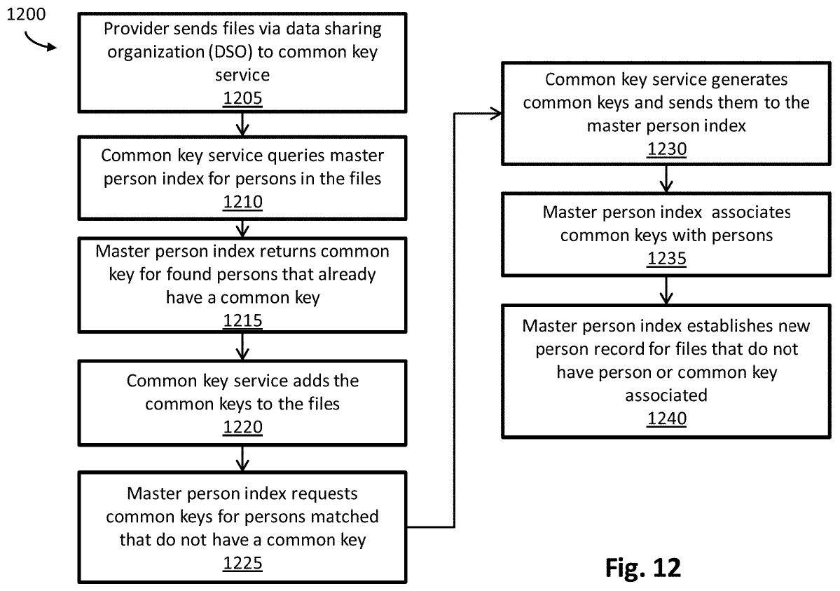

FIG. 12 is a flow diagram illustrating a method 1200 for updating files using a common key service in accordance with an embodiment of the present disclosure. In alternative embodiments, fewer, additional, and/or different operations may be performed. Also, the use of a flow diagram is not meant to be limiting with respect to the order of operations performed. In an operation 1205, a healthcare provider sends files via a DSO device (e.g., the health organization computing devices 510 or the provider computing devices 515) to a common key service. In an operation 1210, the common key service queries an MPI for persons that match persons represented in the files sent from the DSO. In an operation 1215, the MPI returns a common key for any matched individuals in the MPI database that match the persons in the files sent from the DSO. In the operation 1215, the persons matched have already been assigned a common key. In an operation 1220, the common key service adds the common keys attributed to the matched persons to the files. In an operation 1225, the MPI requests common keys for persons matched that do not already have a common key assigned. In an operation 1230, the common key service generates common keys for those persons and sends the common keys to the MPI. In an operation 1235, the MPI associates the generated common keys with the person found in the MPI but previously not yet assigned a common key. In an operation 1240, the MPI establishes a new person record for each of the persons from the files that do not yet have a matched person in the MPI or an associated common key. The method demonstrated in FIG. 12 may be utilized to intake and process large amounts of files and records that apply to many varying persons.

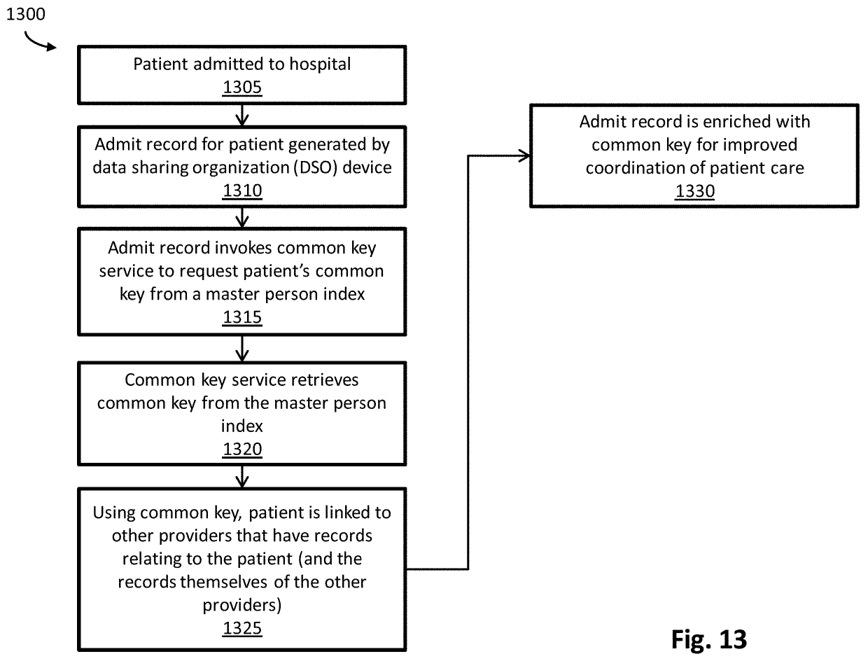

FIG. 13 is a flow diagram illustrating a method 1300 for utilizing a common key service in conjunction with a patient's hospital visit in accordance with an embodiment of the present disclosure. In alternative embodiments, fewer, additional, and/or different operations may be performed. Also, the use of a flow diagram is not meant to be limiting with respect to the order of operations performed. In an operation 1305, a patient is admitted to a hospital. In an operation 1310, an admit record for the patient is generated by a DSO device (e.g., a health organization computing device 510 or a provider computing device 515). In an operation 1315, the admit record and the DSO device invokes a common key service to request the patient's common key from an MPI. In an operation 1320, the common key service retrieves the patient's common key from the MPI. In alternative embodiments, the patient's common key may be generated similar to the common keys discussed above with respect to FIGS. 7 and 8. In an operation 1325, the common key service links the common key to link the patient to other providers that have records relating to that patient (and to the patient's records possessed by the other providers). In an operation 1330, the admit record is enriched with the common key for improved coordination of patient care. In another embodiment, the admit record may also be enriched with other information, such as the linked other providers and other provider records identified in the operation 1325.

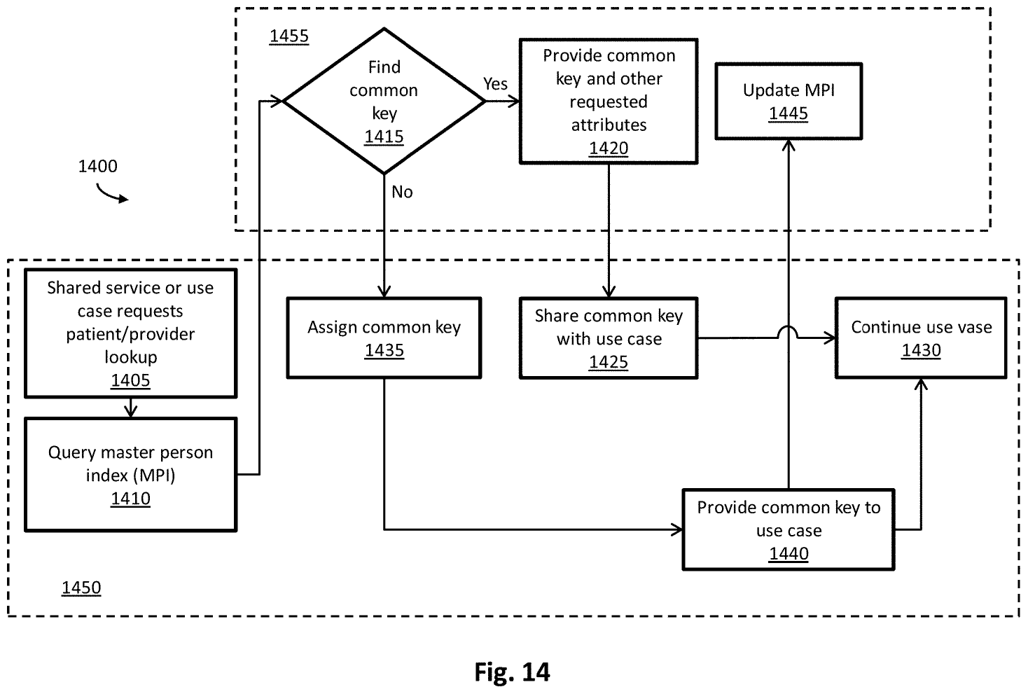

FIG. 14 is a flow diagram illustrating a method 1400 for utilizing a common key service in accordance with an embodiment of the present disclosure. In alternative embodiments, fewer, additional, and/or different operations may be performed. Also, the use of a flow diagram is not meant to be limiting with respect to the order of operations performed. The method 1400 shows steps that may be performed by an MPI 1455, and steps that may be performed by a common key service 1450. In an operation 1405, a shared service or use case requests a patient and/or provider lookup from the common key service 1450. In an operation 1410, the common key service 1450 queries the MPI 1455. In an operation 1415, the MPI 1455 determines whether the patient and/or provider has a common key. If the patient and/or provider does have a common key, the MPI 1455 provides the common key and any other requested attributes (such as demographic information or other records) to the common key service 1450. In an operation 1425, the common key service 1450 in turn provides that information and the common key to the requestor, shared service, use case, etc. In an operation 1430, the common key service 1450 then continues the use case, shared service, etc. In other words, the shared service, use case, etc. utilizes the provided information and/or common key for a purpose for which the information and/or common key was requested, such as obtaining or updating a record. In an operation 1435, the common key service 1450 assigns a common key because the MPI 1455 did not find a common key in the operation 1415. In an operation 1440, the generated common key is provided to the use case, shared service, requestor, etc. In an operation 1445, the generated common key is provided to the MPI 1455, so that the MPI 1455 may be updated with the generated common key. In the operation 1445, the generated common key is associated in the MPI 1455 with the patient and/or provider that the common key has been generated for, such that the common key may be subsequently associated with that patient and/or provider. In the operation 1430, the common key service 1450 then continues the use case, shared service, etc. In other words, the shared service, use case, etc. utilizes the provided information and/or common key for a purpose for which the information and/or common key was requested, such as obtaining or updating a record.

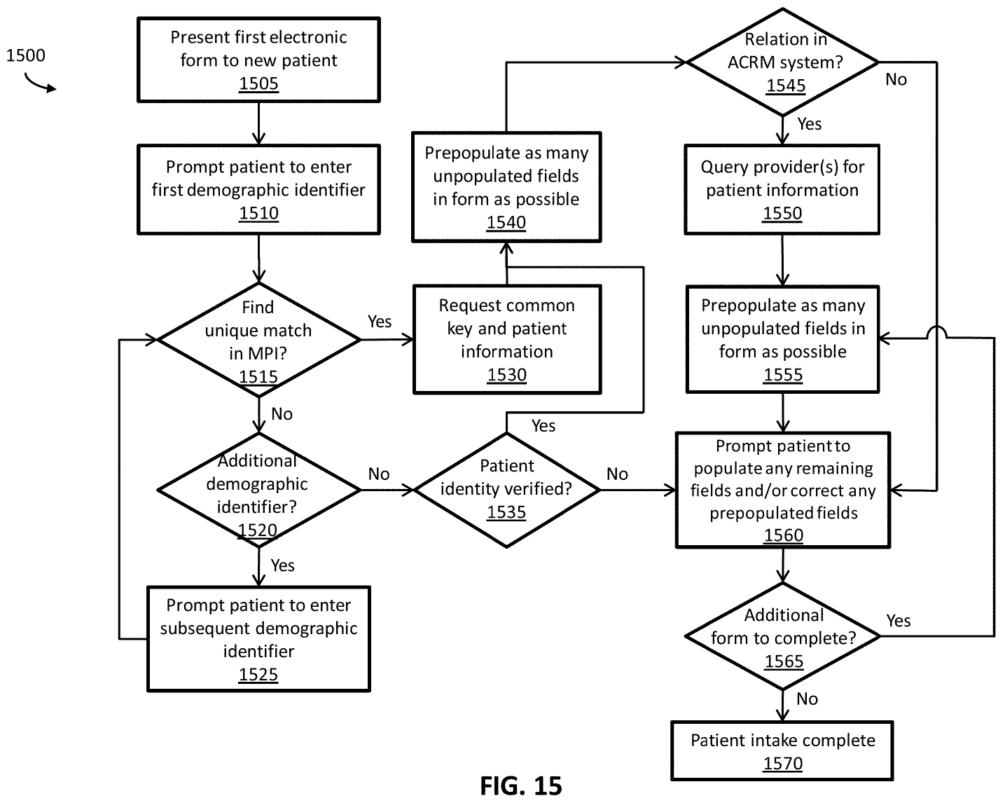

FIG. 15 is a flow diagram illustrating a new patient's intake process 1500 at a health organization or a provider in accordance with an embodiment of the present disclosure. In alternative embodiments, fewer, additional, and/or different operations may be performed. Also, the use of a flow diagram is not meant to be limiting with respect to the order of operations performed. In an operation 1505, a DSO device (e.g., a health organization computing device 510 or a provider computing device 515) presents, on its display, the new patient with an electronic version of a first of one or more intake or consent forms. In an operation 1510, the DSO device prompts the patient to enter a first demographic identifier, which may for example be the patient's first name. Once the patient enters the first demographic identifier, in an operation 1515, the DSO device invokes a common key service to determine if there is a unique match for the patient in an MPI. If there is no match, in an operation 1520, the DSO device determines if there is an additional demographic identifier (e.g., middle initial, last name, zip code, date of birth, gender, last four digits of social security number, phone number, email address, etc.) that the patient may enter. If so, in operation 1525, the DSO device prompts the patient to enter a subsequent demographic identifier. The DSO device then may repeat the operations 1515-1525 until either a unique match is found in the MPI for the patient at operation 1515 or there is a determination that there is no additional demographic identifier for the patient to enter at operation 1520.

If a unique match is found at the operation 1515, at an operation 1530, the DSO device requests, from the common key service, the patient's common key and all additional information that the MPI has on the patient. The MPI typically stores all demographic identifiers of the patients. If there is no additional demographic identifier to be entered, the DSO device invokes, at an operation 1535, an identity proofing service to determine if the identity of the patient may be verified. An identity proofing service is described in U.S. patent application Ser. Nos. 14/642,092 and 14/949,395, and may employ knowledge-based authentication and/or optional biometric identification. When either the patient's common key and MPI information are received at the operation 1530 or if the patient's identity is verified by the identity proofing service at the operation 1535, the DSO device prepopulates, at an operation 1540, as many unpopulated fields in the electronic form as possible using information from the MPI or the identity proofing service.

At an operation 1545, the DSO device invokes an ACRM system (e.g., ACRM system 505) that is used to provide an ACRS to determine, using either the patient's common key from the operation 1530 or the patient's verified identity from the operation 1535, if there is any known relationship between the patient and other providers where information about the patient may be stored. For every provider with which the patient has a relationship, the DSO device queries, at an operation 1550, the provider for information about the patient. The operation 1550 may involve retrieving an electronic address for the provider from a provider directory utility and invoking an intelligent query broker (as described in U.S. patent application Ser. No. 15/855,319, which is incorporated herein in its entirety) to query the provider at its electronic address for information about the patient.

Once additional information about the patient is received from other provider(s), the DSO device prepopulates as many remaining unpopulated fields in the electronic form as possible at an operation 1555. At an operation 1560, the DSO device prompts the patient to populate any remaining fields and/or correct any prepopulated fields. The operation 1560 may also be reached from the operation 1535 when the identity of the patient may not be verified by the identity proofing service.

At an operation 1565, the DSO device determines if there is any additional electronic intake or consent form for the patient to populate. If there is any additional electronic form, the DSO device loops back to operation 1555 and prepopulates as many fields as possible in the new form using information obtained from the MPI, the patient's identity verification, or provider(s) with the which patient has a relationship. Then, at the operation 1560, the DSO device prompts the patient to populate any remaining fields. If there is no additional form for the patient to populate at the operation 1565, the patient intake process 1500 is completed at an operation 1570. The patient intake process 1500 thus allows a patient to create intake forms once and enter every piece of information once, allowing secure communication and accurate sharing of the patient's information regardless of geographical location of health organizations or providers.

At this point it should be noted that intaking a patient at a provider in accordance with the present disclosure as described above may involve the processing of input data and the generation of output data to some extent. This input data processing and output data generation may be implemented in hardware or software. For example, specific electronic components may be employed in a computer server or similar or related circuitry for implementing the functions associated with intaking a patient at a provider in accordance with the present disclosure as described above. Alternatively, one or more processors operating in accordance with instructions may implement the functions associated with intaking a patient at a provider in accordance with the present disclosure as described above. If such is the case, it is within the scope of the present disclosure that such instructions may be stored on one or more non-transitory processor readable storage media (e.g., a magnetic disk or other storage medium), or transmitted to one or more processors via one or more signals embodied in one or more carrier waves.

The embodiments herein may be suitably implemented on conventional computing devices, for example, computer workstations, on Internet-based applications, on optical computing devices, neural computers, biological computers, molecular computing devices, and other devices. As may be appreciated by those skilled in the art, the present invention, in short, may be implemented on any system, automaton, and/or Turing machine.

An automaton is herein described as a mechanism that is relatively self-operating and designed to follow a predetermined sequence of operations or respond to encoded instructions. A Turing machine is herein described as an abstract expression of a computing device that may be realized or implemented on an infinite number of different physical computing devices. Examples of systems, automatons and/or Turing machines that may be utilized in performing the process of the present invention include, but are not limited to: electrical computers (for example, an International Business Machines (IBM) personal computer); neuro-computers (for example, one similar to the “General Purpose Neural Computer” described in U.S. Pat. No. 5,155,802, issued to Paul H. Mueller, on Oct. 13, 1992); molecular computers (for example, one similar to the “Molecular Automata Utilizing Single or Double-Strand Oligonucleotides” described in U.S. Pat. No. 5,804,373, issued to Allan Lee Schweiter et al., on Sep. 8, 1998); biological computers (for example, one similar to the biological computer presented by Ehud Shapiro, of the Computer Science and Applied Mathematics Department at the Weizman Institute of Science (Rehovot, Israel), at the Fifth International Meeting on DNA-Based Computers); and optical computers. For purposes of simplicity, such devices hereinafter are referred to as computers, as is commonly understood in the art. But, the embodiments disclosed herein are not limited being applied to such devices and may be accomplished upon any system or collection of systems capable of providing the features and functions identified herein. For example, the embodiments disclosed herein may be applied to devices such as neurosynaptic computers, application specific computers (or application specific integrated circuits, sometimes referred to as ASICs), software-defined hardware, domain-specific systems on a chip, processors devoted specifically to artificial intelligence-related tasks, or any computer, processor or chip with a special architecture.

It should be understood that the systems described above may provide multiple ones of any or each of those components and these components may be provided on either a standalone machine or, in some embodiments, on multiple machines in a distributed system. The systems and methods described above may be implemented as a method, apparatus or article of manufacture using programming and/or engineering techniques to produce software, firmware, hardware, or any combination thereof. In addition, the systems and methods described above may be provided as one or more computer-readable programs embodied on or in one or more articles of manufacture. The term “article of manufacture” as used herein is intended to encompass code or logic accessible from and embedded in one or more computer-readable devices, firmware, programmable logic, memory devices (e.g., EEPROMs, ROMs, PROMs, RAMs, SRAMs, etc.), hardware (e.g., integrated circuit chip, Field Programmable Gate Array (FPGA), Application Specific Integrated Circuit (ASIC), etc.), electronic devices, a computer readable non-volatile storage unit (e.g., CD-ROM, floppy disk, hard disk drive, etc.). The article of manufacture may be accessible from a file server providing access to the computer-readable programs via a network transmission line, wireless transmission media, signals propagating through space, radio waves, infrared signals, etc. The article of manufacture may be a flash memory card or a magnetic tape. The article of manufacture includes hardware logic as well as software or programmable code embedded in a computer readable medium that is executed by a processor. In general, the computer-readable programs may be implemented in any programming language, such as LISP, PERL, C, C++, C#, PROLOG, or in any byte code language such as JAVA. The software programs may be stored on or in one or more articles of manufacture as object code.

While various embodiments of the methods and systems have been described, these embodiments are exemplary and in no way limit the scope of the described methods or systems. Those having skill in the relevant art can effect changes to form and details of the described methods and systems without departing from the broadest scope of the described methods and systems. Thus, the scope of the methods and systems described herein should not be limited by any of the exemplary embodiments and should be defined in accordance with the accompanying claims and their equivalents.

1. A system for managing data privacy, comprising:
a consent management server comprising a memory, a hardware processor coupled to the memory, and a set of instructions stored in the memory and configured to be executed by the hardware processor, wherein the hardware processor is configured to:
receive, from a patient computing device, consent information including a first patient identifier, a plurality of health organization identifiers, and an indication that a patient corresponding to the first patient identifier consents to sharing of health information by a plurality of health organizations corresponding to the plurality of health organization identifiers;
reconcile the received consent information with stored consent information in a consent record in a database, the consent record being in the form of a blockchain including a set of linked entries, wherein each entry includes information corresponding to the patient, the stored consent information, and a pointer indicating one or more entities that have the patient's consent to receive or transport the patient's health information;
update, in the database, the consent record based on the reconciled consent information by updating at least one entry in the blockchain;
receive, from a data sharing organization, a query including a second patient identifier to determine whether the data sharing organization has permission to share health information related to the patient;
determine that the data sharing organization has permission to share health information related to the patient, based on the query and the consent record;
generate a response to the query based on the determination that the data sharing organization has permission to share health information related to the patient; and
transmit, via a computer network, the response to the data sharing organization.

2. The system of claim 1, wherein the hardware processor is further configured to:
generate information for a graphical user interface comprising a plurality of interface elements including at least a first field corresponding to identification information for the patient and a second field corresponding to a health organization authorized by the patient to share health information related to the patient; and
transmit, the information for the graphical user interface to a computing device of the patient to cause the computing device of the patient to display the graphical user interface.

3. The system of claim 2, wherein the hardware processor is further configured to:
receive, from the patient computing device, a response entered via the first field and the second field of the graphical user interface, the response corresponding to the consent information; and
update the consent record based on the response.

4. The system of claim 3, wherein the hardware processor is further configured to:
generate information for a second graphical user interface comprising an interface element including at least a third field corresponding to a withdrawal of consent for the health organization to share health information related to the patient; and
transmit the information for the second graphical user interface to the computing device of the patient to cause the computing device of the patient to display the second graphical user interface.

5. The system of claim 4, wherein the hardware processor is further configured to:
receive, from the patient computing device, a second response entered via the third field of the second graphical user interface; and
update the consent record to indicate withdrawal of consent for the health organization to share health information related to the patient, based on the second response.

6. The system of claim 1, wherein the hardware processor is further configured to determine that the data sharing organization has permission to share health information related to the patient by determining a first match between the first patient identifier and the second patient identifier and a second match between the data sharing organization and at least one of the plurality of health organization identifiers.

7. The system of claim 1, wherein the hardware processor is further configured to determine that the data sharing organization has permission to share health information related to the patient by determining that an expiration date of the consent record has not passed.

8. The system of claim 1, wherein the hardware processor is further configured to reconcile the received consent information with the stored consent information by:
identifying a first portion of the received consent information that is redundant with respect to the stored consent information; and
discarding the first portion of the received consent information, wherein the reconciled consent information comprises the stored consent information and a remaining portion of the received consent information that does not include the first portion of the received consent information.

9. The system of claim 1, wherein the hardware processor is further configured to reconcile the received consent information with the stored consent information by:
identifying a first portion of the received consent information that is redundant with respect to the stored consent information and a remaining portion of the received consent information not including the first portion of the received consent information;
discarding the remaining portion of the received consent information, wherein the reconciled consent information comprises the first portion of the received consent information that is redundant with respect to the stored consent information.

10. A method for managing data privacy, comprising:
receiving, by a hardware processor from a patient computing device, consent information including a first patient identifier, a plurality of health organization identifiers, and an indication that a patient corresponding to the first patient identifier consents to sharing of health information by a plurality of health organizations corresponding to the plurality of health organization identifiers;
reconciling, by the hardware processor, the received consent information with stored consent information in a consent record in a database, the consent record being in the form of a blockchain including a set of linked entries, wherein each entry includes information corresponding to the patient, the stored consent information, and a pointer indicating one or more entities that have the patient's consent to receive or transport the patient's health information;
updating, by the hardware processor and in the database, the consent record based on the reconciled consent information by updating at least one entry in the blockchain;
receiving, by the hardware processor and from a data sharing organization, a query including a second patient identifier to determine whether the data sharing organization has permission to share health information related to the patient;
determining, by the hardware processor, that the data sharing organization has permission to share health information related to the patient, based on the query and the consent record;
generating, by the hardware processor, a response to the query based on the determination that the data sharing organization has permission to share health information related to the patient; and
transmitting, by the hardware processor and via a computer network, the response to the data sharing organization.

11. The method of claim 10, further comprising:
generating, by the hardware processor, information for a graphical user interface comprising a plurality of interface elements including at least a first field corresponding to identification information for the patient and a second field corresponding to a health organization authorized by the patient to share health information related to the patient;
transmitting, by the hardware processor, the information for the graphical user interface to a computing device of the patient to cause the computing device of the patient to display the graphical user interface.

12. The method of claim 11, further comprising:
receiving, by the hardware processor and from the patient computing device, a response entered via the first field and the second field of the graphical user interface, the response corresponding to the consent information; and
updating, by the hardware processor, the consent record based on the response.

13. The method of claim 12, further comprising:
generating, by the hardware processor, information for a second graphical user interface comprising an interface element including at least a third field corresponding to a withdrawal of consent for the health organization to share health information related to the patient;
transmitting, by the hardware processor, the information for the second graphical user interface to the computing device of the patient to cause the computing device of the patient to display the second graphical user interface.

14. The method of claim 13, further comprising:
receiving, by the hardware processor and from the patient computing device, a second response entered via the third field of the second graphical user interface; and
updating, by the hardware processor, the consent record to indicate withdrawal of consent for the health organization to share health information related to the patient, based on the second response.

15. The method of claim 10, wherein determining that the data sharing organization has permission to share health information related to the patient comprises determining, by the hardware processor, a first match between the first patient identifier and the second patient identifier and a second match between the data sharing organization and at least one of the plurality of health organization identifiers.

16. The method of claim 10, wherein determining that the data sharing organization has permission to share health information related to the patient comprises determining, by the hardware processor, that an expiration date of the consent record has not passed.

17. A method for managing data privacy, comprising:
generating, by a hardware processor, a data structure in the form of a blockchain including a set of linked entries, wherein each entry includes a patient identifier corresponding to a patient, information associated with a plurality of attributes of the patient, and an identification of at least one healthcare provider associated with the patient;
receiving, by the hardware processor, information corresponding to a health event involving the patient;
reconciling, by the hardware processor, the received information with the data structure;
updating, by the hardware processor, the data structure to modify at least one of the plurality of attributes of the patient, based on the health event;
determining, by the hardware processor, whether the patient has provided consent for health information to be delivered to the at least one healthcare provider;
responsive to a determination that the patient has provided consent for the health information to be delivered to the at least one healthcare provider:
determining, by the hardware processor, a delivery method preference for the at least one healthcare provider; and
transmitting, by the hardware processor and via a computer network, information corresponding to the patient identifier and the plurality of attributes to the at least one healthcare provider.

18. The method of claim 17, further comprising transmitting, by the hardware processor, information corresponding to the health event to the at least one healthcare provider, responsive to the determination that the patient has provided consent for the health information to be delivered to the at least one healthcare provider.

19. The method of claim 17, further comprising determining, by the hardware processor, whether the patient has provided consent for a health information organization to receive and transport the health information, prior to transmitting the information corresponding to the patient identifier and the plurality of attributes to the at least one healthcare provider.

20. The method of claim 17, further comprising:
identifying, by the hardware processor, a care team associated with the patient, the care team including a plurality of healthcare providers;
identifying, by the hardware processor and for each of the plurality of healthcare providers included in the care team, a respective electronic address; and
transmitting, by the hardware processor and to each of the respective electronic addresses of the plurality of healthcare providers, a request for health information related to the patient.

21. The method of claim 20, further comprising:
receiving, by the hardware processor and from at least a subset of the plurality of healthcare providers, responses to the request for health information related to the patient; and
updating, by the hardware processor, the data structure to modify at least one of the plurality of attributes of the patient, based on the responses.

22. The method of claim 17, further comprising modifying, by the hardware processor, the data structure to indicate that the patient has provided consent for the health information to be delivered to the at least one healthcare provider.
